(19) United States
(12) Patent Application Publication
(10) Pub. No.: US 2025/0263693 A1
(43) Pub. Date: Aug. 21, 2025

(54) Guide RNA Constructs for Therapeutic Gene Editing

(71) Applicant: Minghong Zhong, Sellersville, PA (US)

(72) Inventor: Minghong ZHONG, Sellersville, PA (US)

(21) Appl. No.: 19/084,983

(22) Filed: Mar. 20, 2025

(51) Int. Cl. C12N 15/10; C12N 9/22; C12N 15/11

(52) U.S. Cl. CPC C12N 15/1082; C12N 9/226; C12N 15/11; C12N 15/111; C12N 2310/20; C12N 2310/31; C12N 2310/531

(57) ABSTRACT

This invention provides guide RNA (gRNA) constructs designed to enhance safety and precision in therapeutic gene editing across RNA-guided systems. The constructs incorporate a non-nucleotide linker near the middle of the spacer sequence, reducing truncated spacer impurities (e.g., n−1 variants) during production by ligating short RNA segments (e.g., 10 nt and 22 nt)—minimizing off-target risks in gene editing therapies. Additionally, a DNA restriction enzyme cleavage site near the spacer's internal end enables excision of a short RNA fragment (e.g., 32 nt for spCas9) for precise spacer purity analysis via LC-MS or electrophoresis. These features ensure robust GMP production and quality control, overcoming limitations of conventional gRNAs and long RNAs (>160 nt) used in diverse editing platforms. Applicable to CRISPR-based and other RNA-guided methods, the constructs maintain or enhance activity, offering a scalable, safe solution for therapeutic gene editing.

### INCORPORATION BY REFERENCE OF SEQUENCE LISTING

The contents of the sequence listing text named “Guide_construct_2025_0414.xml”, which was created on Apr. 14, 2025, and 48,425 bytes in size, are incorporated herein by reference in its entirety.

### TECHNICAL FIELD OF THE INVENTION

The present invention relates to constructs of guide RNAs for uses in gene editing medicine, in particular, relates to guide RNA constructs incorporated with a non-nucleotide linker close to the middle of the spacer sequence of a guide RNA to minimize the truncated spacer sequences (missing one or more internal nucleotides) in production, each of which binds to a wrong DNA region and presents catastrophic risks for the patients. The present invention further relates to constructs of guide RNAs incorporated with a cleavage site of a DNA restriction enzyme close to the internal end of the spacer of a guide RNA, which provides a short guide RNA fragment for accurate analysis of its spacer purity of the guide RNA product after restriction DNA cleavage. The short spacer fragment enables precise and accurate quantitation of truncated spacer impurities to ensure robust GMP production, quality control and release of safe drug substances for therapeutic gene editing.

### BACKGROUND OF THE INVENTION

CRISPR-Cas9 is directed by a guide RNA (e.g., sgRNA) to its targeting site, and the sgRNA is a vital drug substance of in vivo CRISPR therapeutics. However, sgRNA (˜100 nt) presents unprecedented chemistry, manufacturing and control (CMC) challenges. The available commercial scales are at grams with overall isolated yields of single digits (<6%), and the best purity available in the industry is below 50% by LC-MS as reported in spite of reported above 80% purity by HPLC methods, which do not resolve impurities even with lengths of 5 nt shorter. The total ion chromatogram (TIC) data of the full-length product (FLP) peak of commercial products are not accessible to public, if they exist. It should be noted, truncated (n−1) impurities are an assembly of many molecule species, which invalidates quality control methods such as HPLC methods using a reference standard of a specific sequence which cannot sufficiently represent most of truncated molecules or RNA-seq because each single molecule species may not be abundant enough besides the limits of the method such as biases and artifacts in RNA-to-cDNA conversion. Importantly, many truncated products and products of invalid sequences (missing one nucleotide but inserting another one at a different position) are biologically active leading to guide-dependent catastrophic off-targets, though nucleotide(s) missed or inserted within the guide scaffold are not expected to cause off-targets. The synthesis errors of long RNAs increase with their lengths, while nucleotides of the spacer for a Cas9 are added last. A truncated spacer binds to a wrong DNA region and presents catastrophic risks for the patients. It is pivotal to determine the actual off-target causing truncated spacers for better assessment of risks-benefits of treatments, but a reliable quality control method is lacking.

Furthermore, there are rarely any vendors capable of providing chemically modified RNAs longer than 160 nt often used in prime editing and their quality control is even more challenging. There is an urgent need of quality control methods for long guide RNAs, in particular, the spacer purities.

Chemically ligated guide RNA (lgRNA) is our distinct proprietary intellectual property to enable state-of-the-art precise CRISPR gene editing (See, e.g., U.S. Pat. No. 10,059,940B2, the entire disclosures of which are incorporated herein by reference.). LgRNA is a single molecule guide RNA comprising short oligonucleotide segments joined by one or more non-nucleotide Linkers (nNt-Linker). One example is an lgRNA incorporated with a nNt-Linker between a crRNA and a tracrRNA (both shortened) by a chemical ligation. The crRNA segment of an lgRNA is 32 nt long, and such short RNA segments of high quality with refined chemical modifications are routinely manufactured in kilograms in industry. LgRNA provides single-spacer guide products with potential scales of kilograms, and we expected lgRNA to be a critical component of CRISPR therapeutics, including prime editing. The ultra purity of the spacer in LgRNA is ensured by the ultra-purity of the spacer fragment. However, we have still observed the coelutions of minor truncated impurities in ion-pair reverse HPLC separations of the 32 nt segment, due to their close structure similarity, though complete resolution can be very well achieved with a denaturing polyacrylamide gel electrophoresis (PAGE).

In this invention, we disclose guide RNA constructs incorporated with a non-nucleotide linker in the spacer resulting in both unexpectedly improved activity and safety by further relieving the separation challenges with even shorter RNA segments (i.e., 10 nt and 22 nt) and we further disclose guide RNA constructs enabling reliable quality control of long guide RNAs for safe gene editing medicine.

### SUMMARY OF THE INVENTION

The present invention pertains to novel constructs of guide RNAs, their syntheses, and their use in therapeutic gene editing.

In some aspects, the invention provides constructs of guide RNAs for uses in gene editing medicine, in particular, relates to guide RNA constructs incorporated with a non-nucleotide linker close to the middle of the spacer sequence of a guide RNA to minimize the truncated spacer sequences (missing one or more internal nucleotides) in productions by ligation of even shorter RNA segments (e.g., 10 nt and 22 nt).

In some aspects, the invention provides constructs each incorporated with a non-nucleotide linker in the spacer resulting in maintained activity but improved safety due to its high purity.

In some aspects, the invention provides constructs each incorporated with a non-nucleotide linker in the spacer resulting in both improved activity and safety.

In some aspects, the invention provides constructs of guide RNAs incorporated with a cleavage site of a DNA restriction enzyme close to the internal end of the spacer of a guide RNA, which provides a short guide RNA fragment (e.g., 32 nt) for accurate analysis of its spacer purity of the guide RNA product after restriction DNA cleavage. The short spacer fragment enables precise and accurate quantitation of truncated spacer impurities to ensure consistent and robust GMP productions, quality control and release of safe drug substances for therapeutic gene editing.

In some aspects, the invention provides constructs of guide RNAs incorporated with a cleavage site of a DNA restriction enzyme outside and close to the internal end of the spacer of a guide RNA (the end is next to the guide scaffold), wherein the restriction site is a double strand DNA within or next to an RNA stem structure.

In some aspects, the invention provides constructs of guide RNAs incorporated with a cleavage site of a DNA restriction enzyme close to the internal end of the spacer of a guide RNA (the end is next to the guide scaffold), wherein the restriction site is a double strand DNA within or next to an RNA stem structure, wherein one strand of the double strand DNA is fully modified as phosphorothioates.

In some aspects, the invention provides constructs of guide RNAs incorporated with a cleavage site of a DNA restriction enzyme outside and close to the internal end of a guide RNA spacer (the end is next to the guide scaffold), wherein the restriction site is an incorporated single strand DNA (ssDNA) within or next to a single strand RNA region, and the ssDNA can hybridize with a trans-added ssDNA probe for restricted cleavage to result in a short fragment containing the spacer.

### BRIEF DESCRIPTION OF THE DRAWINGS

FIG. 1 and FIG. 2: Guide RNAs (spCas9 as an example) incorporated with a restriction site for quality control of the spacer sequences after a site-selective restriction cleavage.

FIG. 3: LC-MS analysis of a LgRNA after cleavage and strenuous degradation by the restriction enzyme's star effects (ECoRI). The spacer fragment of 32 nt (13.64 min) is marked with an arrow, and also a fragment of 31 nt. An ethanol-precipitated crgRNA pellet was used in ligation step to prepare the guide RNA.

FIG. 4: PAGE analysis of the LgRNA degradation products (by ECoRI) in FIG. 3 (with intensity of bands calculated using a gel image analyzer).

FIG. 5: Mass spectra of total ion chromatogram (TIC) peak at retention time=13.64 min. Mass spectra confirmed it as the spacer fragment, and indicated coelution of truncated spacer impurities.

FIG. 6: Mass spectra of total ion chromatogram (TIC) peak at retention time=13.44 min. Mass spectra confirmed it was a 31 mer, but not the truncated spacer fragment impurities, though much lower percentages of truncated spacer fragment impurities were detected as well. This indicates the use of a reference truncated product for quality control is insufficient.

FIG. 7: In vitro cleavage assay. sg33 is a single guide RNA of spCas9 (sgRNA). lg33-2-3 is a chemically ligated guide RNA (lgRNA) with a non-nucleotide linker replacing the tetraloop nucleotides in sg33. cr7-E1-lg is lg33 inserted with a EcoRI restriction site (with one 5′-g of the recognition site deleted). cr-gfp-E1-lg is a lgRNA (targeting GFP gene) with a EcoRI restriction site. These constructs each with an inserted restriction site are completely active. Sequences (g)aattc and gaattc are joined by a nNt-linker as indicated.

FIG. 8: Guide RNAs (spCas9 as an example) incorporated with a non-nucleotide linker in the spacer (10-10 as an examples). Examples of a minimum-size triazole linker are listed as non-limiting examples.

FIG. 9: In vitro cleavage assay. Sg33 is single guide RNA for spCas9 (sgRNA). lg33 is a chemically ligated guide RNA (lgRNA) with a non-nucleotide linker replacing the tetraloop nucleotides in a sgRNA. cr7-9-tz-11-tz-tr is a lgRNA with a second non-nucleotide linker in the spacer at the position between positions 9 and 10 (numbered from 5′-end, 12gRNA). cr3-10-tz-10-tz-tr is a lgRNA with a second non-nucleotide linker in the spacer at the position between positions 10 and 11 (numbered from 5′-end), i.e., a 12gRNA.

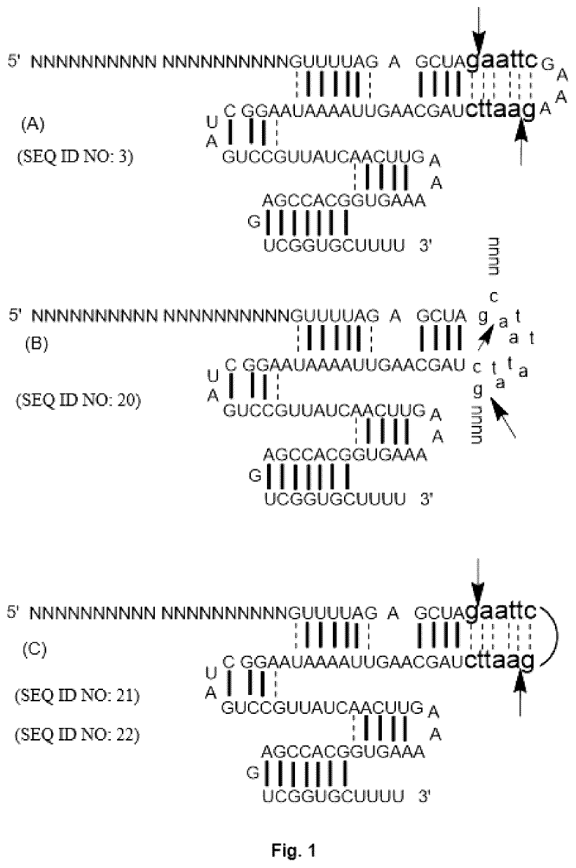

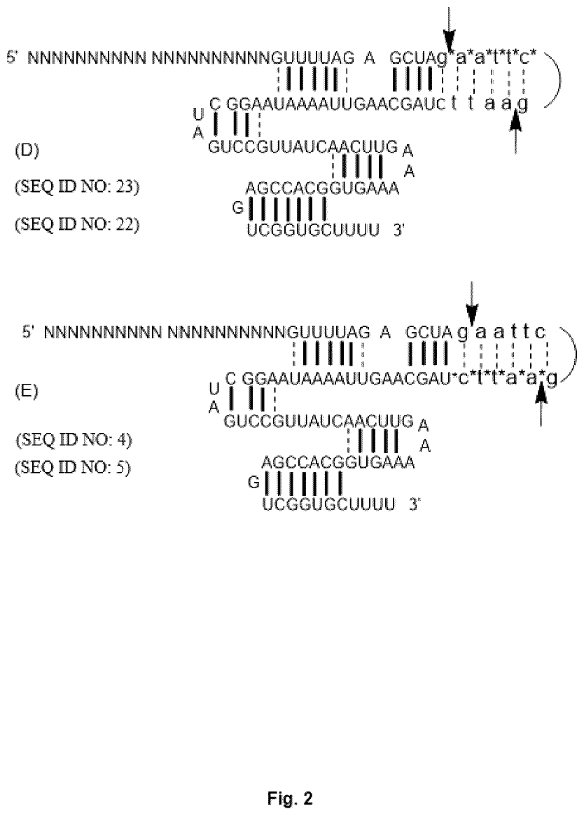

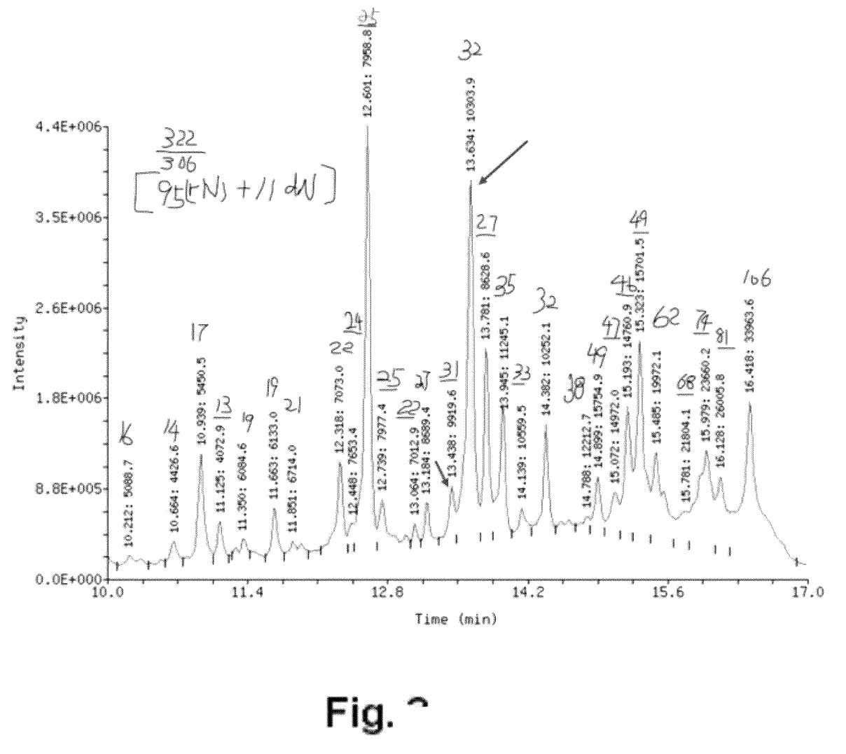

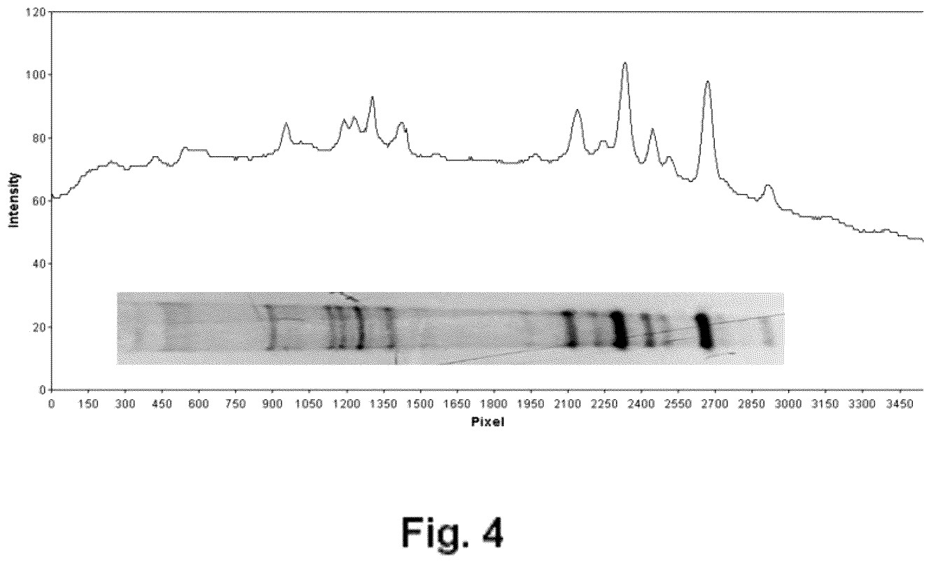

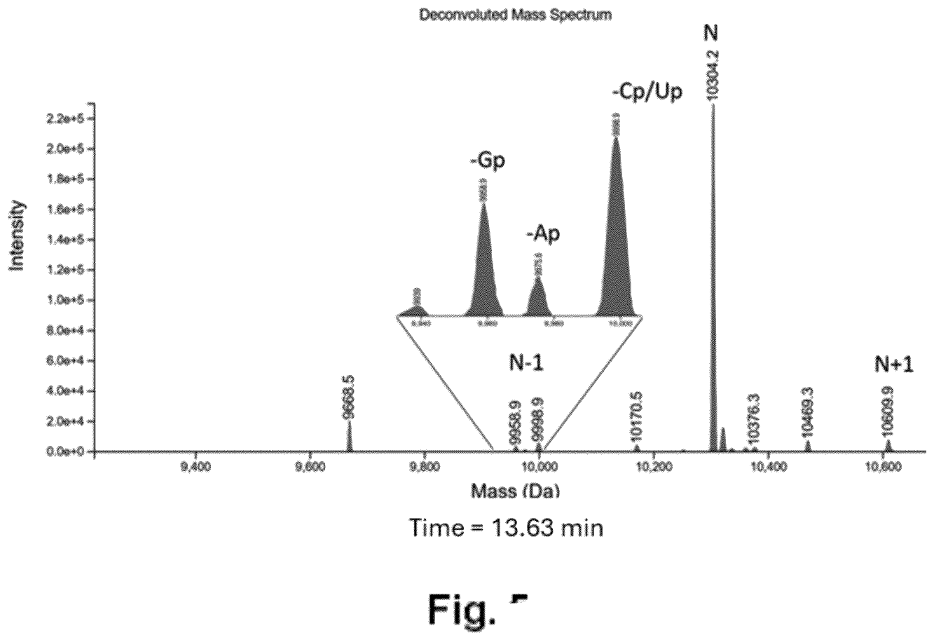

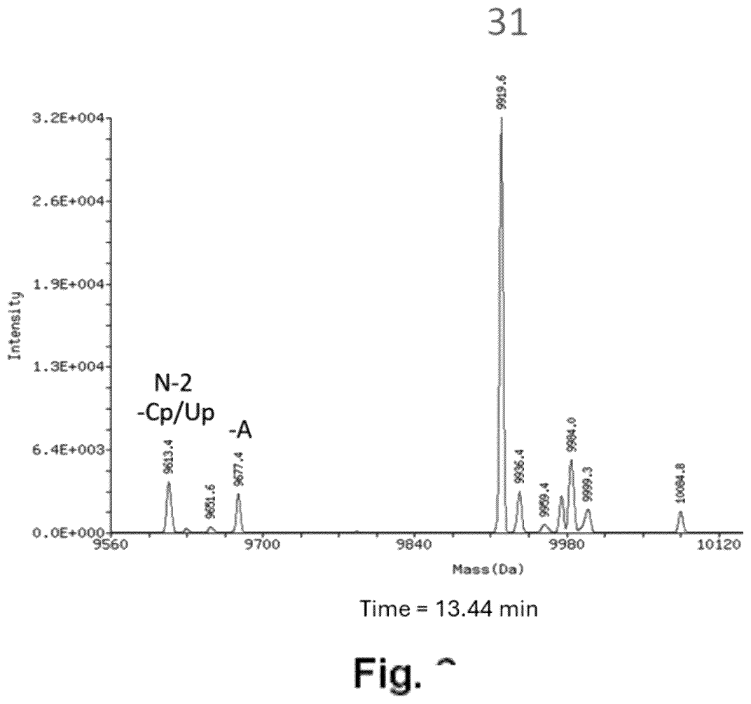

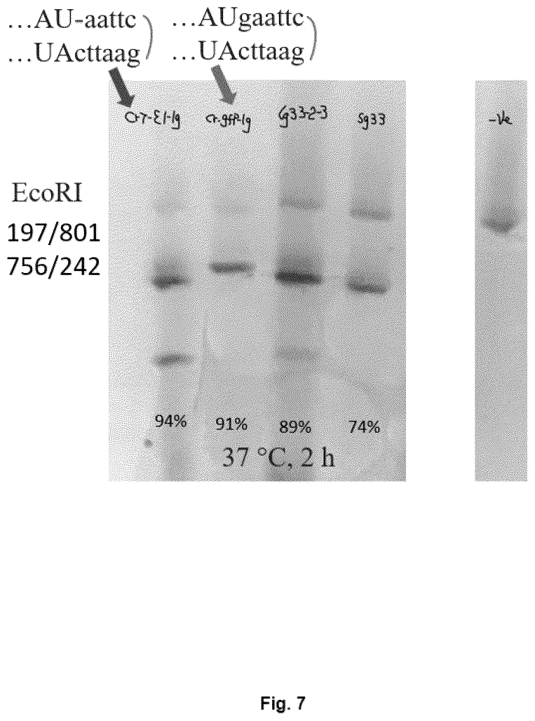

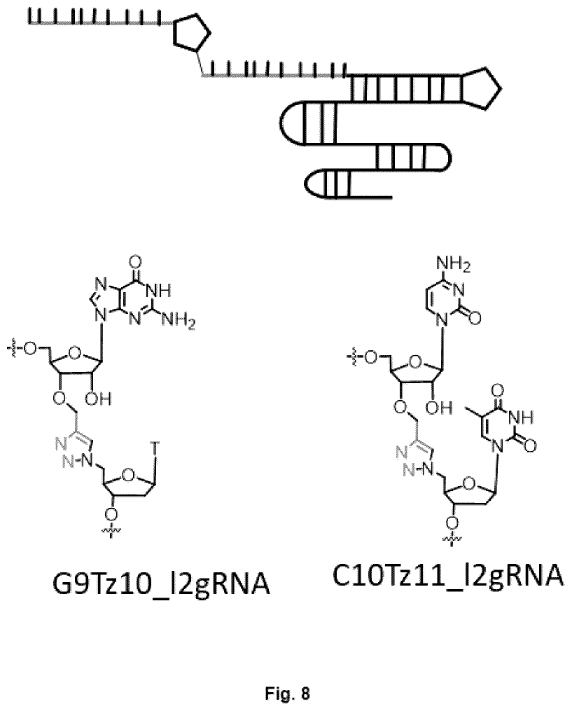

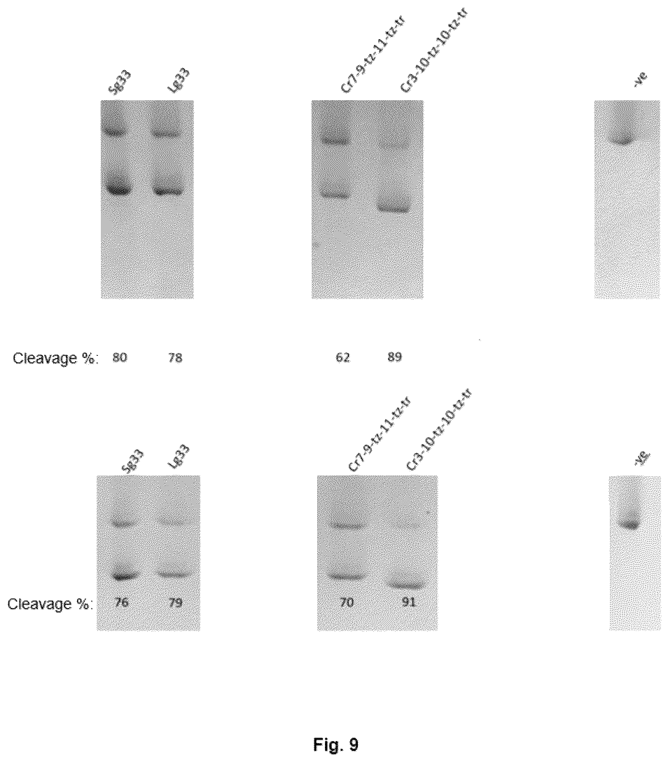

### DETAILED DESCRIPTION OF THE INVENTION

An aspect of the invention is directed to safe gene editing for therapeutic applications with guide RNAs of ultra-pure spacer, comprising: (1) constructs of guide RNAs containing segmented spacers joined by a non-nucleotide linker and (2) incorporation of a restriction site for selective cleavage by a DNA restriction enzyme to form a short RNA fragment enabling accurate determination of truncated spacers with off-target risks.

One embodiment of the invention is insertion of a non-nucleotide linker in the spacer of a guide RNA (sgRNA or lgRNA).

In some embodiments, the insertion is between positions 5 and 6, 6 and 7, 7 and 8, 8 and 9, 9 and 10, 10 and 11, 11 and 12, 12 and 13, 13 and 14, 14 and 15 or 16 and 17 (numbered from 5′-end).

In some embodiments, the inserted non-nucleotide linker maintains the activity of the guide RNA.

In some embodiments, the inserted non-nucleotide linker improves the activity of the guide RNA.

In some embodiments, the non-nucleotide linker in the spacer results in maintained activity but improved safety due to its high purity.

In some embodiments, the non-nucleotide linker in the spacer results in both improved activity and safety.

In some embodiments, the inserted non-nucleotide linker is a chemical moiety formed by conjugation chemistry (ligation).

In some embodiments, the inserted non-nucleotide linker is selected from the group comprising the chemical structures (triazole, tz) between two selected adjacent nucleotides in the spacer:

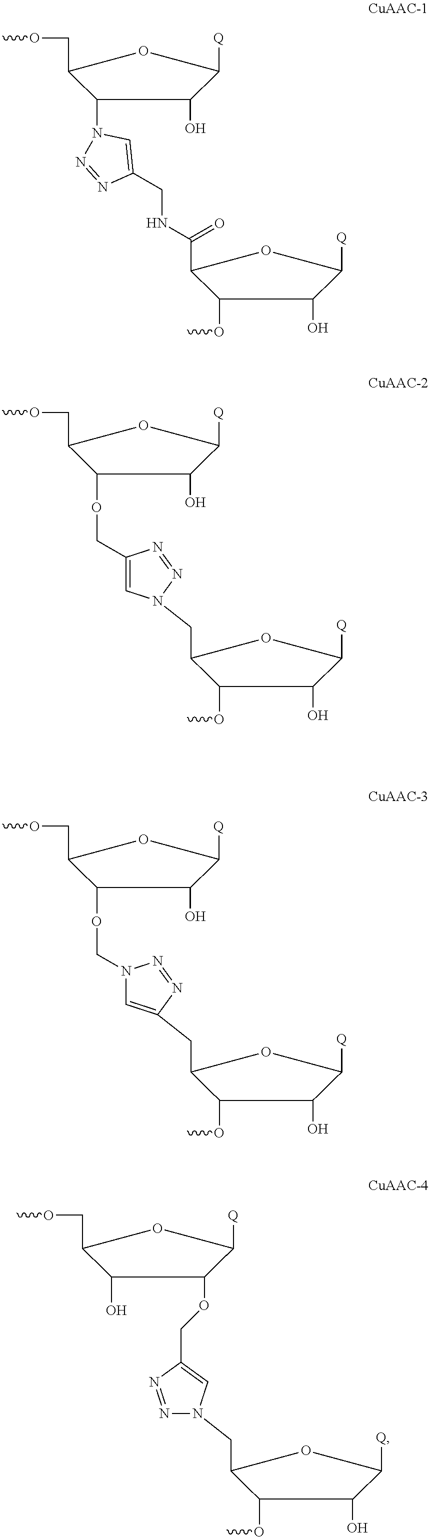

wherein Q is a nucleic acid base, and two Qs can be the same or different, and the sugar moieties are optionally modified (e.g., 2′-deoxy, 2′-MeO, 2′-F, etc.).

In some embodiments, the non-nucleotide linker is inserted between two adjacent nucleotides which are optionally modified:

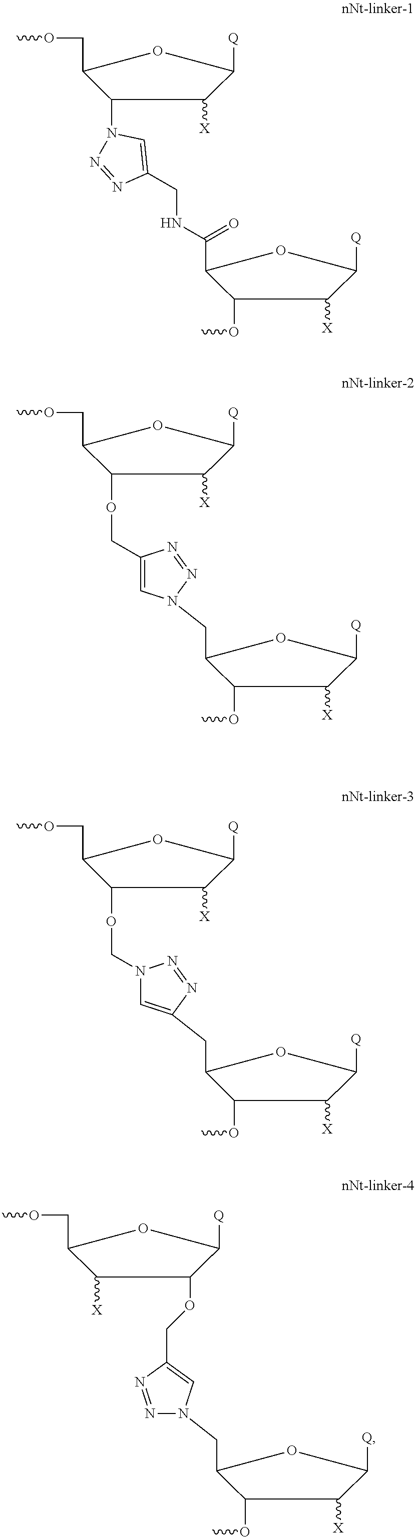

wherein the two Qs are optionally modified nucleic acid bases which can be the same or different, and X is selected from the group consisting of H, F, Cl, low alkyl of C1-C6, OH, OMe, OR, OMOE, NH2 and NHR and can be in either α- or β-configuration, or is covalently linked to 4′-position to form a locked nucleoside, wherein R is an acyl, alkyl, substituted alkyls, or aryl, and the two Xs can be the same or different.

In some embodiments, the constructs of guide RNAs incorporated with a non-nucleotide linker close to the middle of the spacer sequence of a guide RNA to minimize the truncated spacer sequences (missing one or more internal nucleotides) in productions by ligation of short RNA segments (e.g., 10 nt and 22 nt).

One embodiment of the invention is incorporation of restriction site for selective cleavage by a DNA restriction enzyme to form a short RNA fragment enabling accurate determination of truncated spacers with off-target risks.

In some embodiments, the inserted restriction site maintains the activity of the guide RNA. In some embodiments, the inserted restriction site improves the activity of the guide RNA. In some embodiments, the inserted restriction site is selected from the group comprising: non-limiting examples of restriction sites given below together with the corresponding enzymes. The point of cleavage is indicated by a “/”.

| | Enzyme |
|---|---|
| Restriction site | (Source: New England Biolab) |
| AA/CGTT | AclI |
| A/AGCTT | HindIII HindIII-HF ® |
| AAT/ATT | SspI-HF ® |
| /AATT | MluCI |
| A/CATGT | PciI |
| A/CCGGT | AgeI-HF ® |
| A/CGCGT | MluI-HF ® |
| A/CGT | HpyCH4IV |
| A/GATCT | BglII |
| AGC/GCT | AfeI |
| AG/CT | AluI |
| AGG/CCT | StuI |
| AGT/ACT | ScaI-HF ® |
| AT/CGAT | BspDI ClaI |
| ATGCA/T | NsiI NsiI-HF ® |
| AT/TAAT | AseI |
| ATTT/AAAT | SwaI |
| C/AATTG | MfeI-HF ® |
| CAC/GTG | PmlI |
| CAG/CTG | PvuII PvuII-HF ® |
| CA/TATG | NdeI |
| /CATG | FatI |
| CATG/ | NlaIII |
| C/CATGG | NcoI-HF ® NcoI |
| C/CCGGG | TspMI XmaI |
| CCC/GGG | SmaI |
| CCGC/GG | SacII |
| C/CGG | MspI HpaII |
| C/CTAGG | AvrII |
| CCTGCA/GG | SbfI-HF ® |
| CGAT/CG | PvuI-HF ® |
| CG/CG | BstUI |
| C/GGCCG | EagI-HF ® |
| C/GTACG | BsiWI BsiWI-HF ® |
| CGTCTC | BsmBI-v2 |
| C/TAG | BfaI |
| C/TCGAG | PaeR7I XhoI |
| CTGCA/G | PstI PstI-HF ® |
| C/TTAAG | AflII |
| G/AATTC | EcoRI EcoRI-HF ® |
| GAC/GTC | ZraI |
| GACGT/C | AatII |
| GAGCT/C | SacI-HF ® |
| GAG/CTC | Eco53kI |
| GAT/ATC | EcoRV-HF ® EcoRV |
| /GATC | DpnII MboI Sau3 AI |
| GA/TC | DpnI |
| GCATG/C | SphI SphI-HF ® |
| GCCC/GGGC | SrfI |
| GCC/GGC | NaeI |
| G/CCGGC | NgoMIV |
| GCGAT/CGC | AsiSI |
| G/CGC | HinP1I |
| GCG/C | HhaI |
| G/CGCGC | BssHII |
| GC/GGCCGC | NotI NotI-HF ® |
| G/CTAGC | NheI-HF ® |
| GCTAG/C | BmtI-HF ® |
| G/GATCC | BamHI BamHI-HF ® |
| GG/CC | HaeIII |
| GGCCGG/CC | FseI |
| GGCGC/C | PluTI |
| GGC/GCC | SfoI |
| GG/CGCC | NarI |
| G/GCGCC | KasI |
| GG/CGCGCC | AscI |
| G/GGCCC | PspOMI |
| GGGCC/C | ApaI |
| G/GTACC | Acc65I |
| GGTAC/C | KpnI-HF ® |
| GT/AC | RsaI |
| G/TAC | CviQI |
| GTATAC | BstZ17I-HF ® |
| G/TCGAC | SalI SalI-HF ® |
| G/TGCAC | ApaLI |
| GTT/AAC | HpaI |
| GTTT/AAAC | PmeI |
| TAC/GTA | SnaBI |
| T/CATGA | BspHI |
| T/CCGGA | BspEI |
| T/CGA | TaqI-v2 |
| TCG/CGA | NruI-HF ® |
| T/CTAGA | XbaI |
| T/GATCA | BclI BclI-HF |
| TG/CA | HpyCH4V |
| TGC/GCA | FspI |
| TGG/CCA | MscI |
| T/GTACA | BsrGI-HF ® |
| T/TAA | MseI |
| TTAAT/TAA | PacI |
| TTA/TAA | PsiI-v2 |
| TT/CGAA | BstBI |
| TTT/AAA | DraI |

Complete lists of these sites and enzymes can be found from various commercial vendors' catalogs. In some embodiments, the restriction site and enzyme are artificially designed or modified from existing ones for uses equivalent to this invention.

In some embodiments, a guide RNA is incorporated with a cleavage site of a DNA restriction enzyme close to the internal end of the spacer of a guide RNA, which provides a short guide RNA fragment (e.g., 32 nt for spCas9) for accurate analysis of its spacer purity of the guide RNA product after restriction DNA cleavage. The short spacer fragment enables precise and accurate quantitation of truncated spacer impurities to ensure consistent and robust GMP productions, quality control and release of drug substances for safe therapeutic gene editing.

In some embodiments, a guide RNA is incorporated with a cleavage site of a DNA restriction enzyme outside and close to the internal end of the spacer of a guide RNA (the end is next to the guide scaffold), wherein the restriction site is a double strand DNA within or next to an RNA stem structure (an RNA duplex).

In some embodiments, a guide RNA is incorporated with a cleavage site of a DNA restriction enzyme close to the internal end of the spacer of a guide RNA (the end is next to the guide scaffold), wherein the restriction site is a double strand DNA within or next to an RNA stem structure, wherein one strand of the double strand DNA is fully or partially modified as phosphorothioates.

In some embodiments, a guide RNA is incorporated with a cleavage site of a DNA restriction enzyme outside and close to the internal end of the of a guide RNA spacer (the end is next to the guide scaffold), wherein the restriction site is an incorporated single strand DNA (ssDNA) within or next to a single strand RNA region, and the ssDNA can hybridize with a trans ssDNA probe for restricted cleavage to result in a short spacer fragment.

### DEFINITION

The definitions of terms used herein are consistent to those known to those of ordinary skill in the art, and in case of any differences the definitions are used as specified herein instead.

The term “nucleoside” as used herein refers to a molecule composed of a heterocyclic nitrogenous base, containing an N-glycosidic linkage with a sugar, particularly a pentose. An extended term of “nucleoside” as used herein also refers to acyclic nucleosides and carbocyclic nucleosides.

The term “nucleotide” as used herein refers to a molecule composed of a nucleoside monophosphate, di-, or triphosphate containing a phosphate ester at 5′-, 3′-position or both. The phosphate can also be a phosphonate or a phosphoramidate. The oxo in a nucleotide can be replaced by S or CF₂.

The term of “oligonucleotide” (ON) is herein used interchangeably with “polynucleotide”, “nucleotide sequence”, and “nucleic acid”, and refers to a polymeric form of nucleotides of any length, either deoxyribonucleotides or ribonucleotides, or analogs thereof. An oligonucleotide may comprise one or more modified nucleotides, which may be imparted before or after assembly of such an oligonucleotide. The sequence of nucleotides may be interrupted by non-nucleotide components.

The term of “modification” of nucleic acids includes but is not limited to (a) end modifications, e.g., 5′ end modifications or 3′ end modifications, (b) nucleobase (or “base”) modifications, including replacement or removal of bases, (c) sugar modifications, including modifications at the 2′, 3′, and/or 4′ positions, and (d) backbone modifications, including modification or replacement of the phosphodiester linkages. The term “modified nucleotide” generally refers to a nucleotide having a modification to the chemical structure of one or more of the base, the sugar, and the phosphodiester linkage or backbone portions, including nucleotide phosphates. (See, e.g., Ryan et al. US20160289675, the entire disclosure of which is incorporated herein by reference.)

The terms “Z” and “P” refer to the nucleotides, nucleobases, or nucleobase analogs developed by Steven Benner and colleagues as described for example in “Artificially expanded genetic information system: a new base pair with an alternative hydrogen bonding pattern” Yang, Z., Hutter, D., Sheng, P., Sismour, A. M. and Benner, S. A. Nucleic Acids Res. 2006, 34, 6095-101, the contents of which is hereby incorporated by reference in its entirety.

The terms “xA”, “xG”, “xC”, “xT”, or “x (A, G, C, T)” and “yA”, “yG”, “yC”, “yT”, or “y(A, G, C, T)” refer to nucleotides, nucleobases, or nucleobase analogs as described by Krueger et al. in “Synthesis and Properties of Size-Expanded DNAs: Toward Designed, Functional Genetic Systems”; Krueger et al. Acc. Chem. Res. 2007, 40, 141-50, the contents of which is hereby incorporated by reference in its entirety.

The term “Unstructured Nucleic Acid” or “UNA” refers to nucleotides, nucleobases, or nucleobase analogs as described in U.S. Pat. No. 7,371,580, the contents of which is hereby incorporated by reference in its entirety. An unstructured nucleic acid, or UNA, modification is also referred to as a “pseudo-complementary” nucleotide, nucleobase or nucleobase analog (See, e.g., Lahoud et al. Nucl. Acids Res. 1991, 36:10, 3409-19).

The terms “PACE” and “thioPACE” refer to internucleotide phosphodiester linkage analogs containing phosphonoacetate or thiophosphonoacetate groups, respectively. These modifications belong to a broad class of compounds comprising phosphonocarboxylate moiety, phosphonocarboxylate ester moiety, thiophosphonocarboxylate moiety and thiophosphonocarboxylate ester moiety. These linkages can be described respectively by the general formulae P(CR1R2)ₙCOOR and (S)—P(CR1R2)ₙCOOR wherein n is an integer from 0 to 6 and each of R1 and R2 is independently selected from the group consisting of H, an alkyl and substituted alkyl.

The term of “G-clamp” refers to a cytosine analogue capable of clamp-like binding to a guanine in helical nucleic acids by formation of additional hydrogen bonds (Sec, e.g., Lin et al. J. Am. Chem. Soc. 1998, 120, 33, 8531-8532; Wilds et al. Angew. Chem. Int. Ed. 2002, 41, 115-117).

The term of “CRISPR/Cas9” refers to the type II CRISPR-Cas system from Streptococcus pyogenes, Cas9 orthologues and variants. The type II CRISPR-Cas system comprises protein Cas9 and two noncoding RNAs (crRNA and tracrRNA). These two noncoding RNAs were further fused into one single guide RNA (sgRNA). The Cas9/sgRNA complex binds double-stranded DNA sequences that contain a sequence match to the first 17-20 nucleotides of the sgRNA and immediately before a protospacer adjacent motif (PAM). Once bound, two independent nuclease domains (HNH and RuvC) in Cas9 each cleaves one of the DNA strands 3 bases upstream of the PAM, leaving a blunt end DNA double stranded break (DSB).

The term of “off-target effects” refers to non-targeted cleavage of the genomic DNA target sequence by Cas9 in spite of imperfect matches between the gRNA sequence and the genomic DNA target sequence. Single mismatches of the gRNA can be permissive for off-target cleavage by Cas9. Off-target effects were reported for all the following cases: (a) same length but with 1-5 base mismatches; (b) off-target site in target genomic DNA has one or more bases missing (‘deletions’); (c) off-target site in target genomic DNA has one or more extra bases (‘insertions’).

The term of “guide RNA” (gRNA) refers to a synthetic fusion of crRNA and tracrRNA via a tetraloop (GAAA) (defined as sgRNA) or other chemical linkers such as an nNt-Linker (defined as lgRNA), and is used interchangeably with “chimeric RNA”, “chimeric guide RNA”, “single guide RNA” and “synthetic guide RNA”. The gRNA contains secondary structures of the repeat: anti-repeat duplex, stem loops 1-3, and the linker between stem loops 1 and 2 (See, e.g., Nishimasu et al. Cell 2014, 156, 935-949).

The term of “dual RNA” refers to hybridized complex of the short CRISPR RNAs (crRNA) and the trans-activating crRNA (tracrRNA). The crRNA hybridizes with the tracrRNA to form a crRNA:tracrRNA duplex, which is loaded onto Cas9 to direct the cleavage of cognate DNA sequences bearing appropriate protospacer-adjacent motifs (PAM).

The term of “lgRNA” refers to guide RNA (gRNA) joined by chemical ligations to form non-nucleotide linkers (nNt-linkers) between crgRNA and tracrgRNA, or at other sites.

The terms of “dual lgRNA”, “triple lgRNA” and “multiple lgRNA” refer to hybridized complexes of the synthetic guide RNA fused by chemical ligations via non-nucleotide linkers. Dual tracrgRNA is formed by chemical ligation between tracrgRNA1 and tracrgRNA2 (RNA segments of ˜30 nt), and crgRNA (˜30 nt) is fused with a dual tracrgRNA (1-tracrgRNA) to form a triple lgRNA duplex (12gRNA), which is loaded onto Cas9 to direct the cleavage of cognate DNA sequences bearing appropriate protospacer-adjacent motifs (PAM). Each RNA segment can be readily accessible by chemical manufacturing and compatible to extensive chemical modifications.

The term “guide sequence” refers to the about 20 bp sequence within the guide RNA that specifies the target site and is herein used interchangeably with the terms “guide” or “spacer”. The term “tracr mate sequence” may also be used interchangeably with the term “direct repeat(s)”.

The term of “crgRNA” refers to crRNA equipped with chemical functions for conjugation/ligation and is used interchangeably with crRNA in an IgRNA comprising at least one non-Nucleotide linker. The oligonucleotide may be chemically modified close to its 3′-end, any one or several nucleotides, or for its full sequence.

The term of “tracrgRNA” refers to tracrRNA equipped with chemical functions for conjugation/ligation and is used interchangeably with tracrRNA in an lgRNA comprising at least one non-nucleotide linker. The oligonucleotide may be chemically modified at any one or several nucleotides, or for its full sequence.

The term of “the protospacer adjacent motif (PAM)” refers to a DNA sequence immediately following the DNA sequence targeted by Cas9 in the CRISPR bacterial adaptive immune system, including NGG, NNNNGATT, NNAGAA, NAAAC, and others from different bacterial species where N is any nucleotide.

The term of “chemical ligation” refers to joining together synthetic oligonucleotides via an nNt-linker by chemical methods such as click ligation (the azide-alkyne reaction to produce a triazole linkage), thiol-maleimide reaction, and formations of other chemical groups.

The term of “complementary” refers to the ability of a nucleic acid to form hydrogen bond(s) with another nucleic acid sequence by either traditional Watson-Crick or other non-traditional types. Cas9 contains two nuclease domains, HNH and RuvC, which cleave the DNA strands that are complementary and noncomplementary to the 20 nucleotide (nt) guide sequence in crRNAs, respectively.

The term of “Hybridization” refers to a reaction in which one or more polynucleotides form a complex that is stabilized via hydrogen bonding between the bases of the nucleotide residues. The complex may comprise two strands forming a duplex structure, three or more strands forming a multi stranded complex, a single self-hybridizing strand, or any combination of these. A sequence capable of hybridizing with a given sequence is referred to as the “complement” of the given sequence.

The synonymous terms “hydroxyl protecting group” and “alcohol-protecting group” as used herein refer to substituents attached to the oxygen of an alcohol group commonly employed to block or protect the alcohol functionality while reacting other functional groups on the compound. Examples of such alcohol-protecting groups include but are not limited to the 2-tetrahydropyranyl group, 2-(bisacetoxyethoxy)methyl group, trityl group, trichloroacetyl group, carbonate-type blocking groups such as benzyloxycarbonyl, trialkylsilyl groups, examples of such being trimethylsilyl, tert-butyldimethylsilyl, tert-butyldiphenylsilyl, phenyldimethylsilyl, triiospropylsilyl, triisopropylsilyloxymethyl (TOM) and thexyldimethylsilyl, ester groups, examples of such being formyl, (C1-C10) alkanoyl optionally mono-, di- or tri-substituted with (C1-C6) alkyl, (C1-C6) alkoxy, halo, aryl, aryloxy or haloaryloxy, the aroyl group including optionally mono-, di- or tri-substituted on the ring carbons with halo, (C1-C6) alkyl, (C1-C6) alkoxy wherein aryl is phenyl, 2-furyl, carbonates, sulfonates, and ethers such as benzyl, p-methoxybenzyl, methoxymethyl, 2-ethoxyethyl group, etc. The choice of alcohol-protecting group employed is not critical so long as the derivatized alcohol group is stable to the conditions of subsequent reaction(s) on other positions of the compound of the formula and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and G.M. Wuts, T.W. Greene, “Protective Groups in Organic Synthesis”, John Wiley & Sons Inc., Hoboken, New Jersey, 2007, which are hereby incorporated by reference. The related terms “protected hydroxyl” or “protected alcohol” define a hydroxyl group substituted with a hydroxyl protecting group as discussed above.

The term “nitrogen protecting group,” as used herein, refers to groups known in the art that are readily introduced on to and removed from a nitrogen atom. Examples of nitrogen protecting groups include but are not limited to acetyl (Ac), trifluoroacetyl (TFA), isopropyl-phenoxyacetyl or phenoxyacctyl (PAC), Boc, Cbz, benzoyl (Bz), Fluorenylmethyloxycarbonyl (Fmoc), N,N-dimethylformamidine (DMF), trityl, Monomethoxytrityl (MMT), Dimethoxytrityl (DMTr), and benzyl (Bn). See also G. M. Wuts, T. W. Greene, “Protective Groups in Organic Synthesis”, John Wiley & Sons Inc., Hoboken, New Jersey, 2007, and related publications.

The term of “Isotopically enriched” refers to a compound containing at least one atom having an isotopic composition other than the natural isotopic composition of that atom. The term of “Isotopic composition” refers to the amount of each isotope present for a given atom, and “natural isotopic composition” refers to the naturally occurring isotopic composition or abundance for a given atom. As used herein, an isotopically enriched compound optionally contains deuterium, carbon-13, nitrogen-15, and/or oxygen-18 at amounts other than their natural isotopic compositions.

The term “a restriction site” or “restriction recognition sites”, as used herein, are located on a DNA molecule containing specific sequences of nucleotides, which are recognized by restriction enzymes. These are generally palindromic sequences (because restriction enzymes usually bind as homodimers), and a particular restriction enzyme may cut the sequence between two nucleotides within its recognition site, or somewhere nearby. As used herein, “a restriction site” is a short DNA sequence introduced for site-selective cleavage of a guide RNA by a DNA restriction enzyme.

The term “a restriction enzyme”, “restriction endonuclease”, “Rease”, “ENase” or “restrictase” as used herein, refers to an enzyme that cleaves DNA into fragments at or near specific recognition sites within the DNA molecule known as restriction sites.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent known to be useful for, or which has been or is currently being used for the treatment or prevention of a disorder or one or more symptoms thereof.

### Nucleic Acids

The invention relates, in part, to constructs of guide RNAs. All the guides are optionally chemically modified with terminal 2′-methoxy nucleotides and phosphorothioate backbones according to the general standard practice.

One embodiment is constructs of guide RNAs, comprising a non-nucleotide linker in the spacer of the guide:

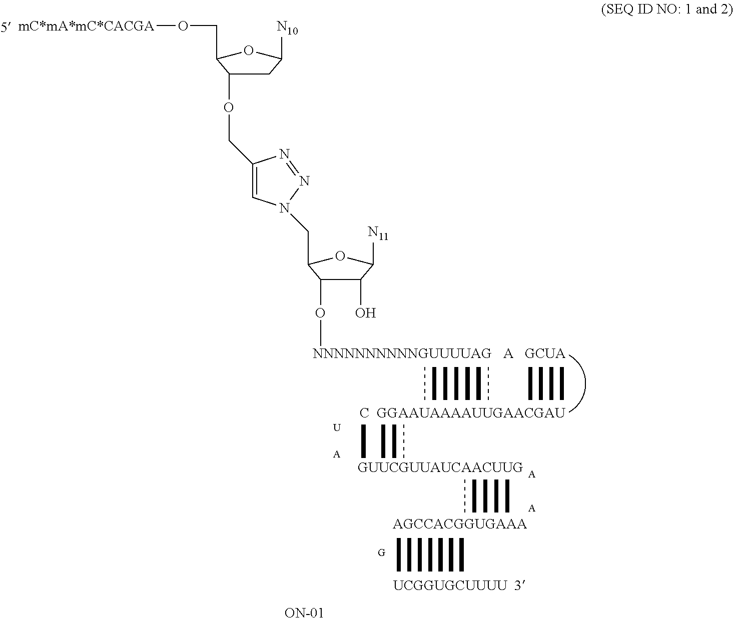

, wherein NNNNNNNNNN₁₀N₁₁NNNNNNNNN represents a spacer and “ [symbol] ” is a non-nucleotide linker.

One embodiment is constructs of guide RNAs comprising a restriction site.

In some embodiments, the guide RNA is a sgRNA with a nucleotide tetraloop joining the crRNA and tracrRNA, fused with a restriction site.

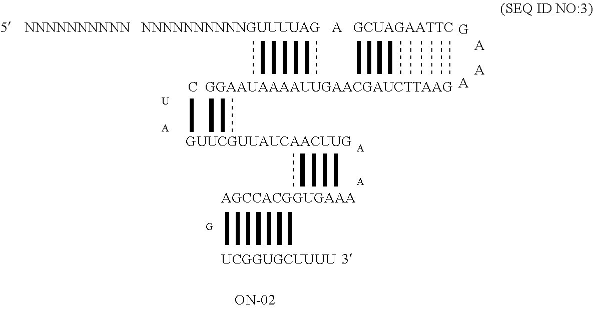

In some embodiments, the guide RNA is a sgRNA with a non-nucleotide linker joining the crRNA and tracrRNA, wherein “*” indicates optional phosphorothioate modifications, fused with a restriction site.

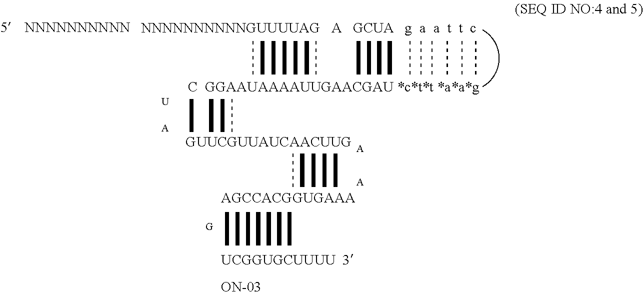

In some embodiments, the guide RNA is a prime editing guide (pegRNA or epegRNA), inserted with a restriction site. The guide RNA can be formed from its corresponding segments by chemical ligations. An example is given below, wherein “ [symbol] ” is either a nucleotide linker or a non-nucleotide linker.

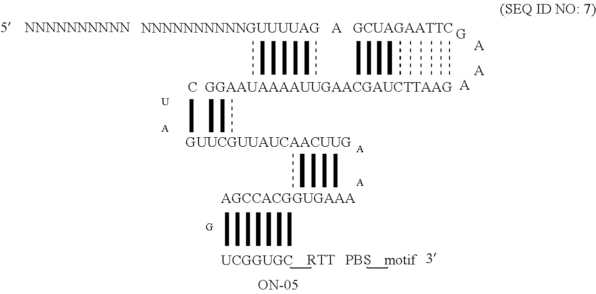

In some embodiments, the guide RNA is a STAR editing guide (segNA) inserted with a restriction site. The guide RNA can be formed from its corresponding segments by chemical ligations. An example is given below, wherein “ [symbol] ” is either a nucleotide linker or a non-nucleotide linker.

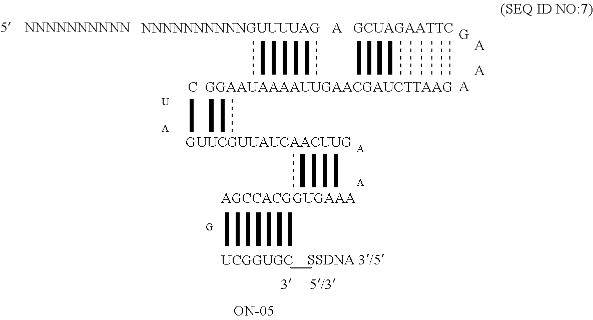

Non-Nucleotide Linkers (nNt-Linker)

An nNt-Linker, formed by chemical ligation, comprises an M core structure of Formula M-1 to M-13 as non-limiting examples:

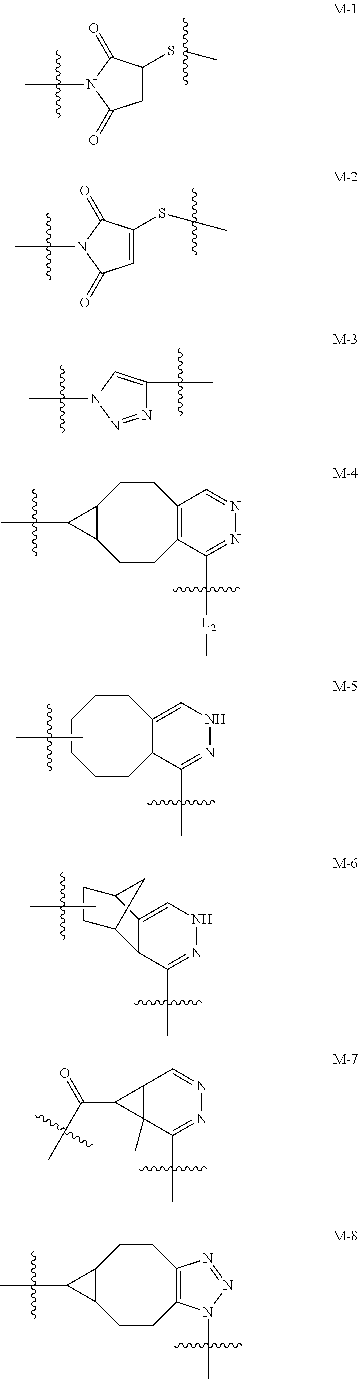

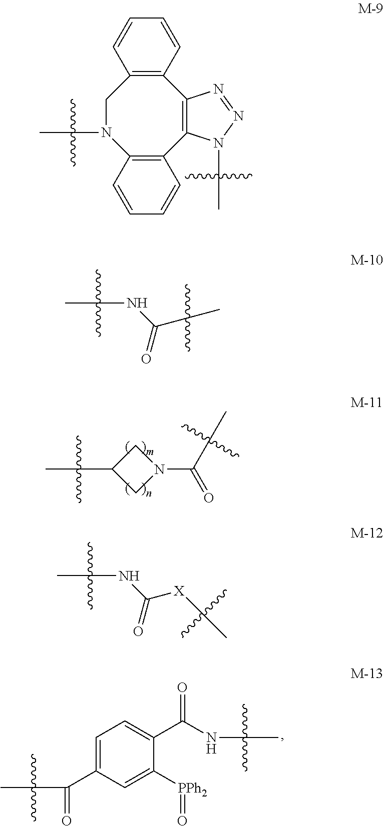

wherein X=O, S, NH, or CH₂, m=0 to 3 and n=0 to 3,
and two L linkers comprising Formula L-1 to L-23 as non-limiting examples:

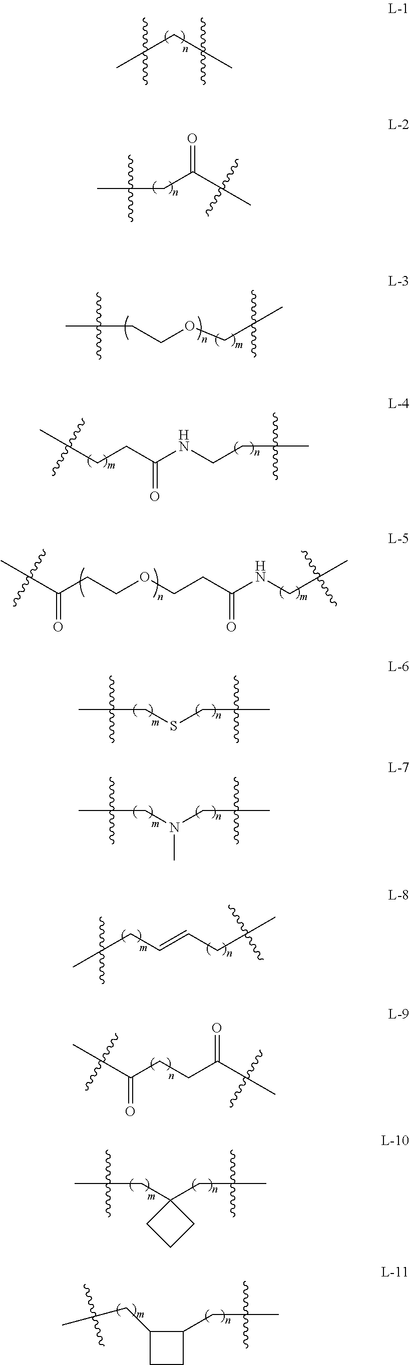

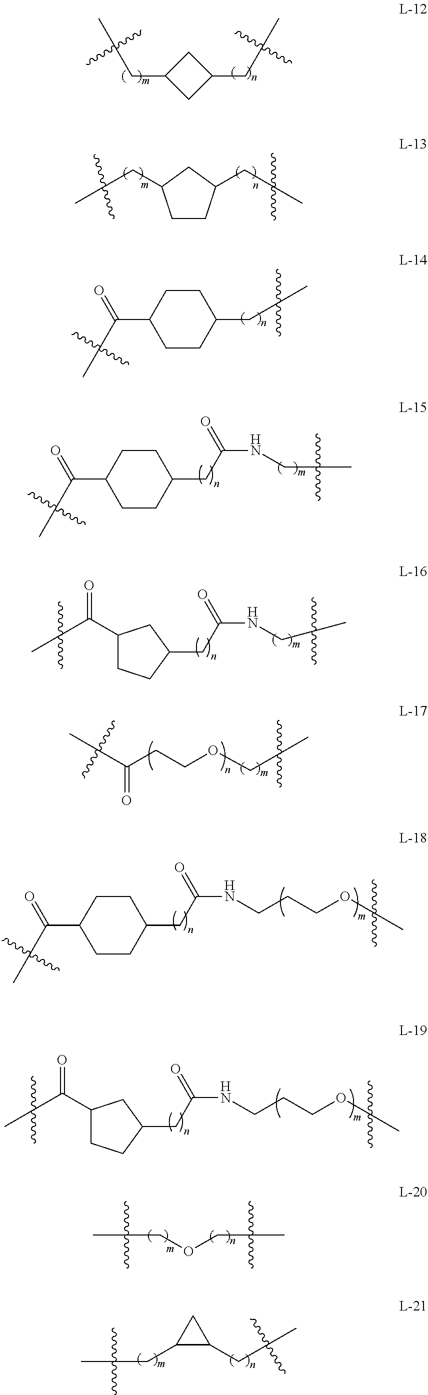

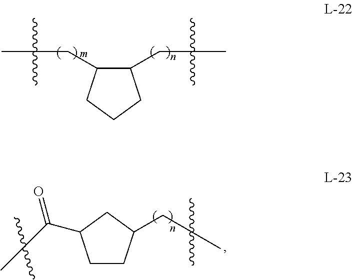

wherein m=0 to 16 and n=0 to 16,
said L linkers and said M core structure are joined as L-M-L. wherein the two L linkers are the same or different, and each L optionally comprises one or more structures of Formula L-1 to L-23 or partial structure(s), and attached to two terminal nucleotides of Formula Nuc-1 to Nuc-18 as non-limiting examples:

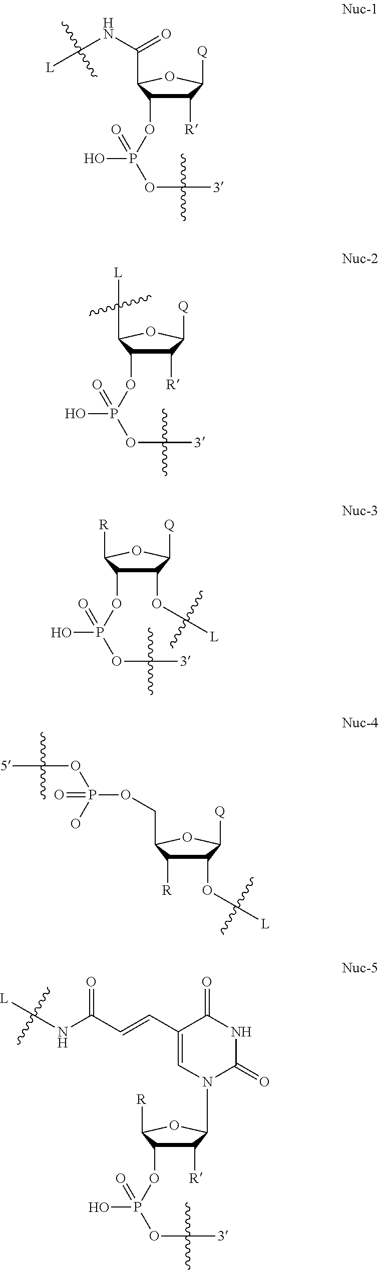

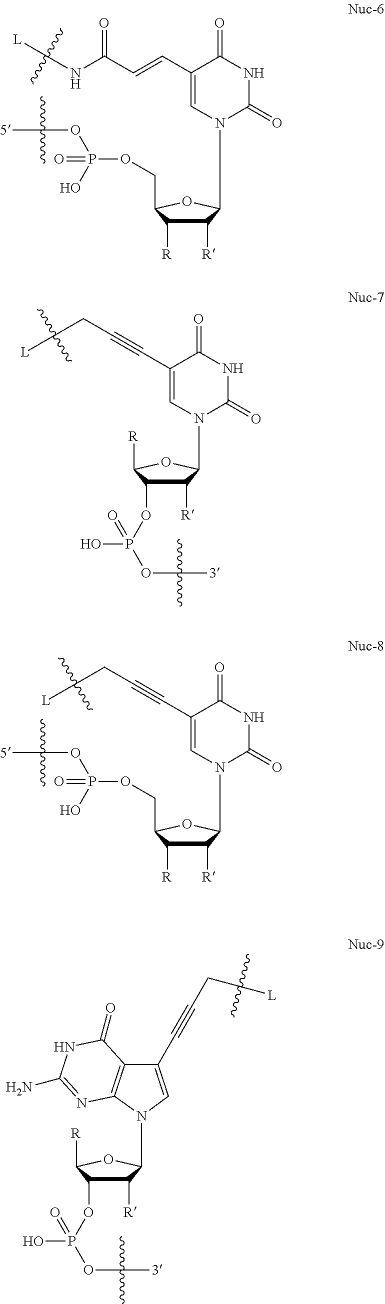

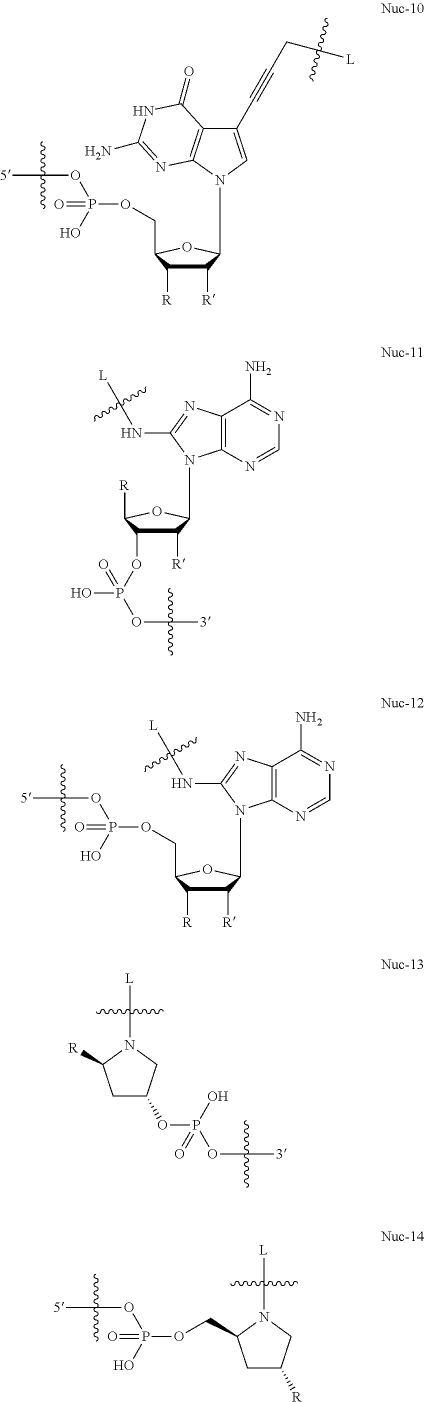

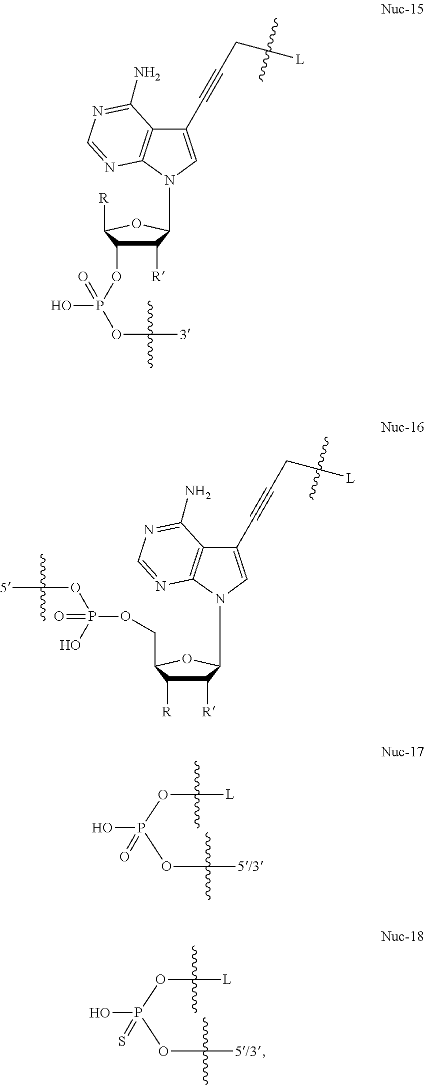

wherein the attached positions are

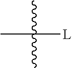

### to L-M-L and

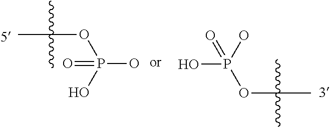 [SMILES: *OP(=O)(O)O ; *OP(=O)(O)O]

to upstream and downstream oligonucleotides, respectively, and wherein R is H, OH,

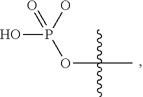 [SMILES: *OP(=O)(O)O]

### CH₂OH,

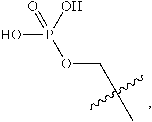 [SMILES: *COP(=O)(O)O]

F, NH₂, OMe, CH₂OMe, OCH₂CH₂OMe, an alkyl, a cycloalkyl, an aryl, or heteroaryl, R′ is H, OH,

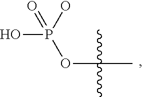 [SMILES: *OP(=O)(O)O]

### CH₂OH,

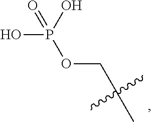 [SMILES: *COP(=O)(O)O]

F, NH₂, OMe, CH₂OMe, OCH₂CH₂OMe, an alkyl, a cycloalkyl, an aryl, or a heteroaryl, and Q is a natural or a non-natural nucleic acid base.

In some embodiments, the M core structure, L, and terminal nucleotides are optionally modified with substituents such as halogen (F, Cl, Br, I), lower alkyl of C₁-C₆, halogenated (F, Cl, Br, I) lower alkyl of C₁-C₆, lower alkenyl of C₂-C₆, halogenated (F, Cl, Br, I) lower alkenyl of C₂-C₆, CN, lower alkynyl of C₂-C₆, halogenated (F, Cl, Br, I) lower alkynyl of C₂-C₆, lower alkoxy of C₁-C₆, halogenated (F, Cl, Br, I) lower alkoxy of C₁-C₆, an optionally substituted aryl, an optionally substituted heteroaryl, an optionally substituted sulfonyl, or optionally substituted acyl, which includes but is not limited to C(═O) alkyl, NR′₂, CN, CO₂H, CO₂R′, CONH₂, CONHR′, CONR′₂, CH═CHCO₂H, or CH═CHCO₂R′, wherein R′ is an optionally substituted alkyl, which includes, but is not limited to, H, an optionally substituted C₁-C₂₀ alkyl, an optionally substituted lower alkyl, an optionally substituted cycloalkyl, an optionally substituted alkynyl of C₂-C₆, an optionally substituted lower alkenyl of C₂-C₆, an optionally substituted aryl, an optionally substituted heteroaryl, an optionally substituted sulfonyl, or optionally substituted acyl, which includes but is not limited to C(═O) alkyl, or alternatively, in the instance of NR′₂, each R′ comprise at least one C atom that are joined to form a heterocycle comprising at least two carbon atoms.

In some embodiments, an nNt-Linker joins the 3′-terminal nucleotide of a crRNA and the 5′-terminal nucleotide of a tracrRNA. In some embodiments, an nNt-Linker joins the 5′-terminal nucleotide of a crRNA and the 3′-terminal nucleotide of a tracrRNA. In some embodiments, an nNt-Linker joins two oligonucleotide segments of tracrRNA.

In some embodiments, one of the two Ls in an nNt-linker (L-M-L) is covalently linked to guide RNA(s), and the other is covalently linked to a PEG polymer, a non-PEG polymer, a ligand for cellular receptors, a lipid, an oligonucleotide, an antibody, a polysaccharide or a peptide.

In some embodiments, one of the two Ls in nNt-linkers (L-M-L) is covalently linked to an exposed amino acid residue of Cas protein such as lysine, serine, and cysteine, and the other is covalently linked to a PEG polymer(s), a non-PEG polymer, a ligand for cellular receptors, a lipid, an oligonucleotide, an antibody, a polysaccharide(s) or a peptide.

In some embodiments, the nNt-linkers between the two nucleotides/nucleosides are represented by the following formulas:

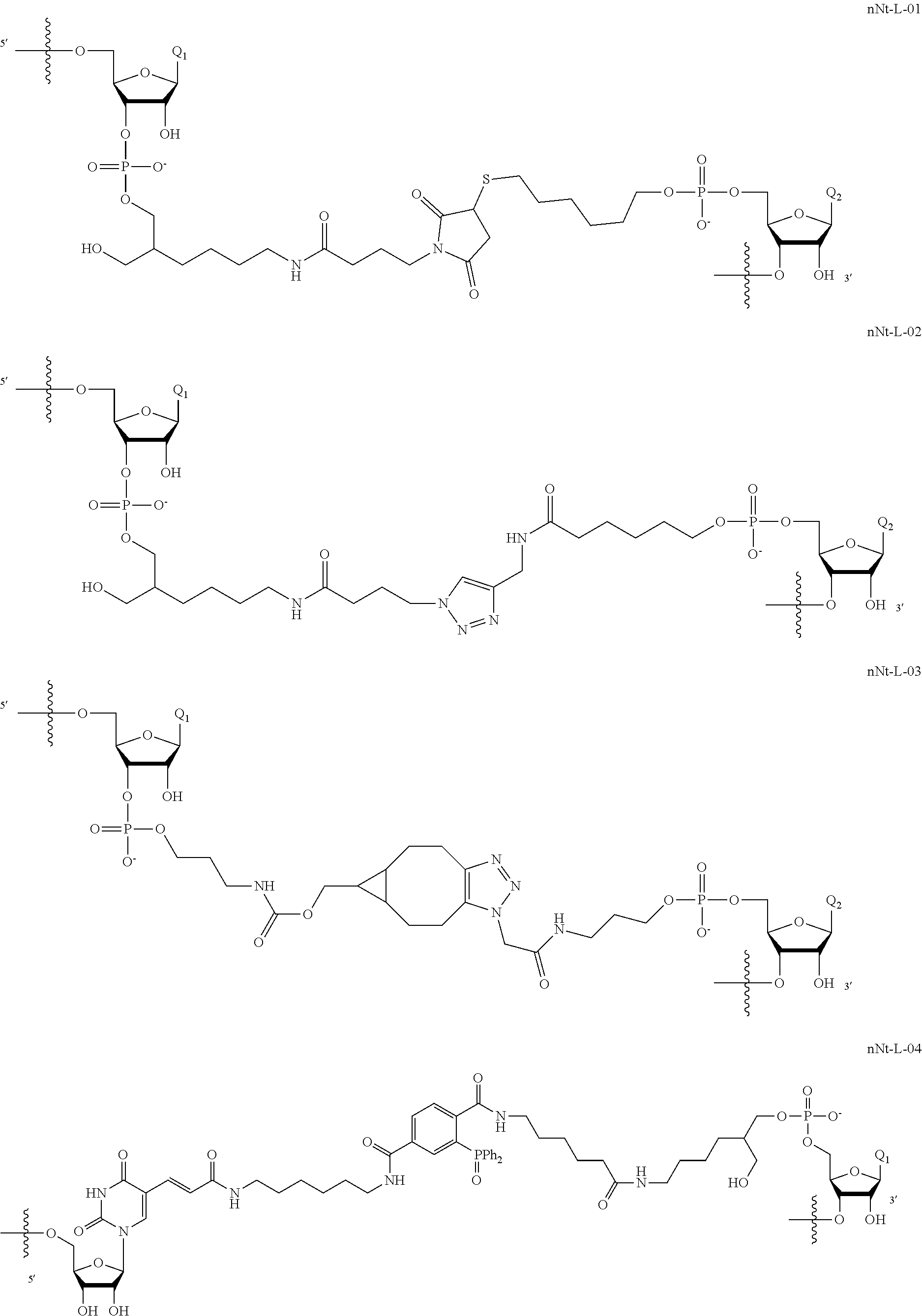

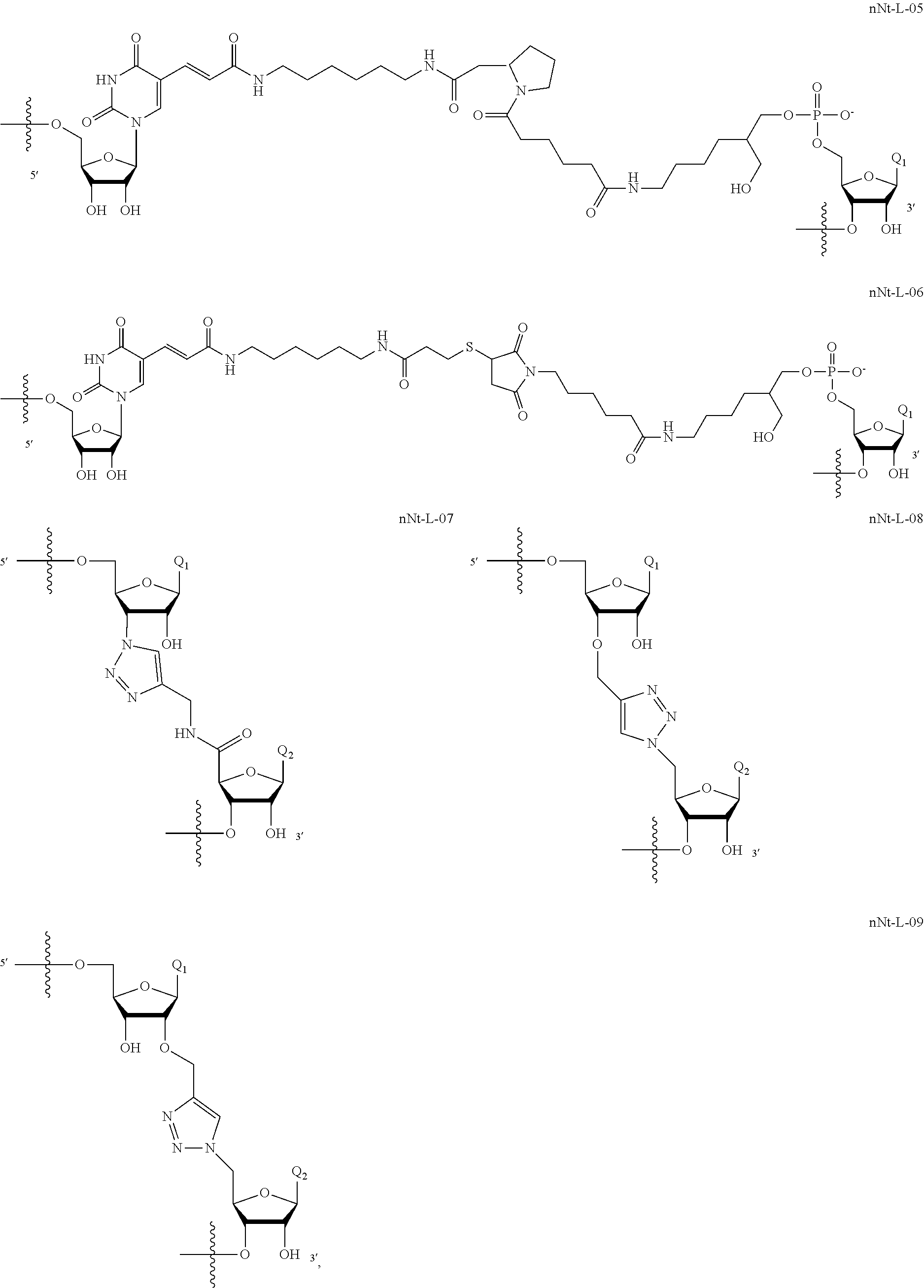

wherein Q₁ and Q₂ are nucleic acid bases and Q1 and Q2 can be the same or different.

### CRISPR Effector Proteins

In some embodiments, CRISPR effector endonuclease is selected from Cas proteins of Type II, Class 2 including Streptococcus pyogenes-derived Cas9 (SpCas9, 4.1 kb), smaller Cas9 orthologues, including Staphylococcus aureus-derived Cas9 (SaCas9, 3.16 kb), Campylobacter jejuni-derived Cas9 (CjCas9, 2.95 kb), Streptococcus thermophilus Cas9 (St1Cas9, 3.3 kb), Neisseria meningitidis Cas9 (NmCas9, 3.2 kb), and many other variants of engineered Cas9 proteins such as SpCas9-HF1, eSpCas9, and HypaCas9, proteins of Type V, Class 2 including Cas12 (Cas12a (Cpf1), Cas12b (C2c1), Cas12c, Cas12e, Cas12g, Cas12h, Cas12i, and etc.) and Cas14, and proteins of Type VI, Class 2 such as Cas13a and Cas13b. The said CRISPR effector protein can be a nickase e.g. nCas9 such as a SpCas9-nickase (D10A or H840A), or a catalytically inactive protein e.g. dCas9 coupled/fused with a protein effector such as a DNA polymerase, FokI, transcription activator(s), transcription repressor(s), catalytic domains of DNA methyltransferase, histone acetyltransferase and deacetylase, reverse transcriptase (prime editor), and nucleic acid deaminases (base editor) at its either N- or C-terminal.

In another embodiment, the said CRISPR effector endonuclease is an artificial one comprising one or more functional domains derived from human.

In yet another embodiment, the said CRISPR effector endonuclease is a class 2 CRISPR Cas protein functionalized by site-directed mutagenesis to introduce orthogonal conjugating sites such as cysteines and remove deleterious conjugating sites (e.g. C80 in SpCas9), and corresponding RNP conjugates are prepared by selective conjugations such as PEGylation of cysteines by maleimide chemistry.

In yet another embodiment, the said CRISPR effector endonuclease is a class 2 CRISPR Cas protein fused with a human DNA or RNA polymerase via a peptide linker. In yet another embodiment, the said CRISPR effector endonuclease is a class 2 CRISPR Cas protein fused with a DNA methyltransferase (e.g., a DNMTI catalytic subunit) via a peptide linker. In yet another embodiment, the said CRISPR effector endonuclease is a class 2 CRISPR Cas protein fused with a DNA polymerase and a DNA methyltransferase. In yet another embodiment, the said CRISPR effector endonuclease is a class 2 CRISPR Cas protein fused with a DNA methyltransferase (e.g., DNMT1) via a peptide linker, wherein the Cas protein is catalytically impaired (e.g., nCas9 (H840A or D10A) and dCas9 (H840A and D10A) derived from Streptococcus pyogenes). In yet another embodiment, the said CRISPR effector endonuclease is a class 2 CRISPR Cas protein fused with a DNA methyltransferase (e.g., DNMT1) and a transcription repressor (e.g., a KRAB domain), wherein the Cas protein is catalytically impaired (e.g., nCas9 (H840A or D10A) and dCas9 (H840A and D10A) derived from Streptococcus pyogenes).

### Other RNA-Guided Effector Proteins

In another embodiment, the effector protein is an endogenous RNA editing or cleaving enzyme, e.g., ADAR and AGO2. The targeting sequence (guide) comprises an internal non-nucleotide linker.

### EXAMPLES

The following examples further illustrate embodiments of the disclosed invention, which are not limited by these examples.

### Example 1: crgRNA

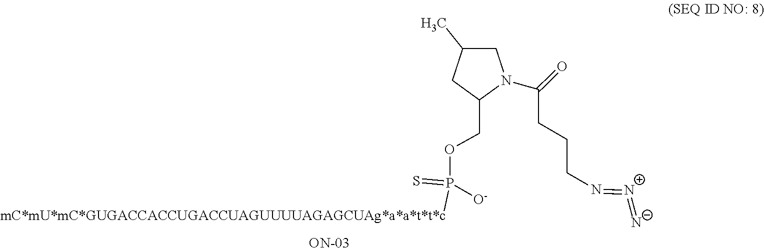

ON-05 was prepared on an Expedite 8909 automated DNA/RNA synthesizer using the standard 1.0 μmole RNA phosphoramidite cycle. 3′-Azido CPG 1000 Å (1 μmole) was packed into an Expedite column. All β-cyanoethyl phosphoramidite monomers were dissolved in anhydrous acetonitrile to a concentration of 0.1 M immediately prior to use. Coupling, capping and oxidation reagents (ChemGenes) were 5-Ethyl-1H-tetrazole (0.45 M in acetonitrile), Cap A (Acetic Anhydride/Pyridine/THF)/Cap B (10% N-Methylimidazole in THF) and iodine (0.02 M Iodine/Pyridine/H₂O/THF), respectively. DDTT solution (0.05 M) in Pyridine/Acetonitrile was used as sulfurizing reagent. Stepwise coupling efficiencies were determined by automated trityl cation conductivity monitoring and in all cases were >97%.

RNA deprotection. Oligonucleotide on solid support was treated with 20% piperidine in DMF at room temperature to suppress the formation of cyanoethyl adducts, then washed with acetonitrile (3×1 mL) and dried with argon.

The oligonucleotide on solid support was exposed to AMA (Ammonium Hydroxide/40% aqueous Methylamine 1:1 v/v) in a sealed vial for 20 min at 65° C. The solution was collected by filtration and the solution was then concentrated till dryness in Savant™ SpeedVac™ vacuum concentrator at room temperature. The resulting white solid was re-dissolved in a 2:2:3 v/v mixture of dry NMP (200 μL), triethylamine (200 μL) and triethylamine trihydrofluoride (300 μL) and heated at 60° C. for 3 h. After cooling down to room temperature, sodium acetate (3 M pH 5.2, 40 μL) and ethanol (1 mL) were added and the RNA was stored for 30 min at −78° C. The RNA was then pelleted by centrifugation (15,850×g, 10 min, 4° C.), the supernatant discarded and the pellet washed twice with 70% ethanol (500 μL). The pellet was then dried in vacuo and used for next step without further purification.

### Example 2: tracrgRNA

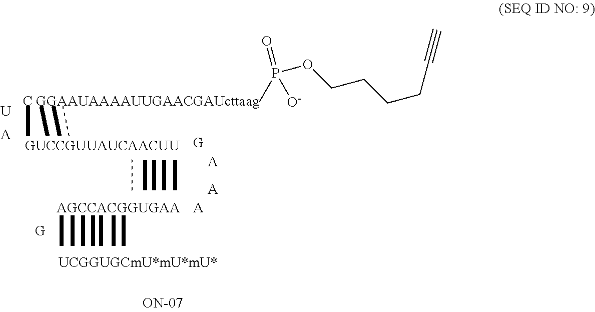

ON-06 was prepared on an Expedite 8909 automated DNA/RNA synthesizer using the standard 1.0 μmole RNA phosphoramidite cycle, fully deprotected and separated as ON-06. 2′-methoxy uridine 3′-lcaa CPG 1000 Å (1 μmole) was used instead. The pellet was then dried in vacuo and used for next step without further purification.

### Example 3: 1 gRNA-eGFP-EcoRI

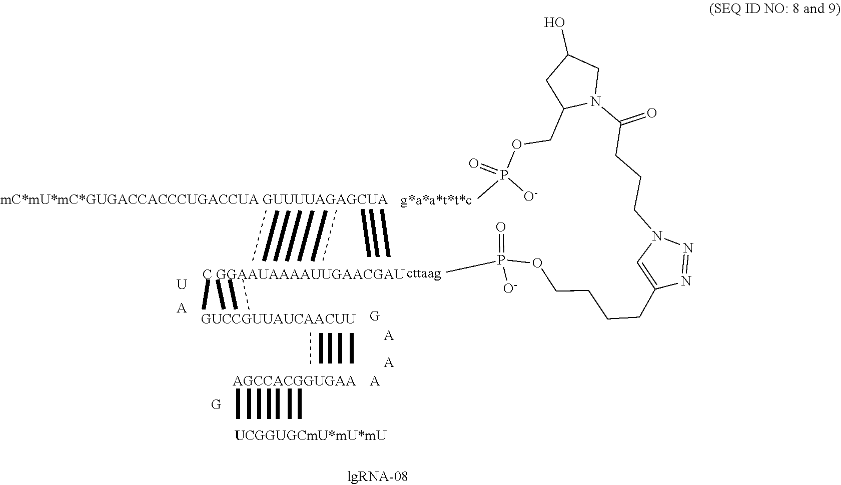

To azide ON-05 pellet (half, <0.49 μmole) and alkyne ON-07 pellet (half, <0.49 μmole) in a stock solution (DMSO/ddH₂O/2 M TEAA, 2:1:0.4, 1700 μL) was added CuSO₄-THPTA (tris-hydroxypropyl triazole ligand) (250 mM, 100 μL), and the resulting light blue solution was deoxygenated by bubbling argon for 10 min. Freshly prepared ascorbic acid in ddH₂O (125 mM, 200 μL) was added, and reaction mixture was further deoxygenated by bubbling argon for 30 min. The reaction mixture was sealed and kept at room temperature for 2 h, and sodium acetate (3 M pH 5.2, 40 μL) and ethanol (1 mL) were added. The resulting RNA suspension was stored for 30 min at −78° C. The RNA was then pelleted by centrifugation (15,850×g, 10 min, 4° C.). The supernatant was discarded and the pellet washed twice with 70% ethanol (500 μL). The pellet was then dried in vacuo at room temperature.

The above oligonucleotide pellet was mixed with gel loading buffer (formamide/ddH₂O 90% v/v, with 10 mM EDTA) and RNA loading dyes (2x) and loaded onto a denaturing 10% polyacrylamide gel (1x TBE buffer containing 7 M urea) and separated at 65 W for 2-3 h. RNA bands were visualized under UV, excised, crushed, soaked in a gel extract buffer (NaCl solution with 2 mM EDTA) overnight at 37° C. with vigorous shaking. The gel was removed by filtration through two consecutive Sep-Pak C18 plus short cartridges, the oligonucleotide solutions were combined, and the final concentration was determined by a NanoDrop spectrophotometer at 260 nm. The solution was concentrated till dryness in vacuo in Savant™ SpeedVac™ vacuum concentrator at room temperature.

### Example 4: In Vitro Cleavage Assay

Recombinant Cas9 protein was purchased from New England BioLabs, Inc. Cas9 and IgRNA or segRNA were preincubated in a 1:1 molar ratio in the cleavage buffer to reconstitute the RNP complex.

The substrate of a dsDNA comprising eGFP gene was dissolved in the cleavage buffer and added to the RNP complex. The reaction mixture was incubated at 37° C. for 1 h, and DNA loading dyes (6x) was added. The resulting mixture was heated at 95° C. for 5 min, cooled to room temperature, and resolved by a 1% Agarose gel.

### Example 5: In Vitro Gene Editing

293/GFP cells (Cell Biolabs) are passaged on the day prior to electroporation.
100 pmol of Cas9-2NLS (or variants) is diluted to a final volume of 5 μL with Cas9 buffer (20 mM HEPES (pH 7.5), 150 mM KCl, 1 mM MgCl₂, 10% glycerol and 1 mM TCEP) and mixed slowly into 5 μL of Cas9 buffer containing 120 pmol of lgRNA or segRNA. The resulting mixture is incubated for 10 min at room temperature to allow RNP formation. 2×10⁻⁵ 293/GFP cells are harvested, washed once in PBS, and resuspended in 20 μL of SF nucleofection buffer (Lonza, Basel, Switzerland). 10 μL of RNP mixture and cell suspension are combined in a Lonza 4 d strip nucleocuvette. Reaction mixtures are electroporated using setting DS150, incubated in the nucleocuvette at room temperature for 10 min, and transferred to culture dishes containing pre-warmed media. Editing outcomes are measured 4 and 7 days post-nucleofection by flow cytometry.

### Example 6: Formation of Cas9-gRNA Complex, Cellular Transfections, and Assays

a. Transfection with cationic lipids (See, e.g., Liu et al. Nature Biotechnology 2015, 33, 73-80, the entire disclosure of which is incorporated herein by reference): Purified synthetic gRNA (lgRNA) or mixture of synthetic gRNAs is incubated with purified Cas9 protein for 5 min, and then complexed with the cationic lipid reagent in 25 μL OPTIMEM. The resulting mixture is applied to the cells for 4 h at 37° C.
b. Transfection with cell-penetrating peptides (See, e.g., Kim et al. Genome Res. 2014, 24: 1012-1019, the entire disclosure of which is incorporated herein by reference): Cell-penetrating peptide (CPP) is conjugated to a purified recombinant Cas9 protein (with appended Cys residue at the C terminus) by drop wise mixing of 1 mg Cas9 protein (2 mg/mL) with 50 μg 4-maleimidobutyryl-GGGRRRRRRRRRLLLL (m9R; 2 mg/mL) (SEQ ID NO: 10) in PBS (pH 7.4) followed by incubation on a rotator at room temperature for 2 h. To remove unconjugated 9 mR, the samples are dialyzed against DPBS (pH 7.4) at 4° C. for 24 h using 50 kDa molecular weight cutoff membranes. Cas9-m9R protein is collected from the dialysis membrane and the protein concentration is determined using the Bradford assay (Biorad).

Synthetic gRNA (lgRNA, 12gRNA or segRNA) or a mixture of synthetic gRNAs is complexed with CPP:gRNA (1 μg) in 1 μl of deionized water is gently added to the C3G9R4LC peptide (9R) in gRNA:peptide weight ratios that range from 1:2.5 to 1:40 in 100 μl of DPBS (pH 7.4). This mixture is incubated at room temperature for 30 min and diluted 10-fold using RNase-free deionized water.

150 μl Cas9-m9R (2 μM) protein is mixed with 100 μl gRNA:9R (10:50 μg) complex and the resulting mixture is applied to the cells for 4 h at 37° C. Cells can also be treated with Cas9-m9R and lgRNA:9R sequentially.

### Example 7: In Vivo Gene Editing by LNP Mediated Delivery LNP Formulations

LNPs are prepared using a NanoAssemblr microfluidic system (Precision Nanosystems) as reported (See, e.g., Qiu et al. Proc Natl Acad Sci U S A. 2021, 118(10): e2020401118, the entire disclosure of which is incorporated herein by reference.). Lipids (6Z,9Z,28Z,31Z)-heptatriaconta-6,9,28,31-tetraen-19-yl 4-(dimethylamino) butanoate (MC-3), DSPC, Cholesterol, and DMG-PEG2000 are dissolved in pure ethanol at a molar ratio of 50% MC-3, 38.5% Cholesterol, 10% DSPC, and 1.5% DMG-PEG2000 with a final MC-3 concentration of 10 mg/mL. Cas9 mRNA and gRNA (lgRNA, 12gRNA or segRNA) are mixed at the appropriate weight ratio in sodium acetate buffer (25 mM, pH 5.2). The RNA solution and the lipid solution are each injected into the NanoAssemblr microfluidic device at a ratio of 3:1, and the device results in the rapid mixing of the two components and thus the self-assembly of LNPs. Formulations are further dialyzed against PBS (10 mM, pH 7.4) in dialysis cassettes overnight at 4° C. The particle size of formulations is measured by dynamic light scattering (DLS) using a ZetaPALS DLS machine (Brookhaven Instruments). RNA encapsulation efficiency is characterized by Ribogreen assay.

In Vivo gene editing by LNP delivery.

The above RNA-LNPs are intravenously injected into mice at a dose of 0.5 mg/kg RNA.

### Example 8: Multiplexing Gene Editing

LgRNAs are synthesized and mixed in an appropriate ratio. The mixture is either delivered with an mRNA or a plasmid or a viral vector encoding a CRISPR Cas protein, or complexes with a Cas protein or a Cas protein conjugate in vitro, and is delivered to target cells as a mixture of RNP complexes.

For in vivo tests, the above mixtures, either alone or with additives such as transfection reagents, are intravenously injected into an animal.

### Example 9: RNA Editing With Segmented gRNAs in ADAR-Expressing 293 Cells

RNA editing in ADAR-expressing 293 cells is performed according to a reported procedure (See, e.g., Merkle et al. Nature Biotech. 2019, 37, 133-138, the entire disclosure of which is incorporated herein by reference.). Segmented gRNA ASO (5 pmol/well unless stated otherwise) and Lipofectamine 2000 (0.75 μL/well) are each diluted with OptiMEM to a volume of 10 μL in separate tubes. After 5 min, the two solutions are mixed and 100 μL cell suspension (5×10⁴ cells) in DMEM plus 10% FBS plus 10 ng/mL doxycycline is added to the transfection mixture inside 96-well plates. Twenty-four hours later, cells are harvested for RNA isolation and sequencing.

### Example 10: Cleavage/Degradation of Guide RNA Constructs Containing a EcoRI Restriction Site

To guide RNA construct (Lg-E1-cr7, 0.3 or 2.5 nmol) in 1x rCutSmart Buffer™ (New England Biolab) was added EcoRI-HF® (20 units/μL, New England Biolab). The reaction mixture was incubated at 37° C. for 2 h or overnight, and 0.5 M EDTA (>50 fold of the reaction) and DNA loading dyes (6x) were added. The resulting mixture was heated at 95° C. for 5 min, cooled to room temperature, and resolved by a 1% Agarose gel.

Alternatively, the reaction for LC-MS analysis was stopped by adding 0.5 M EDTA (>50 fold of the reaction) and heating at 95° C. for 5 min. After being cooled to room temperature, the reaction was diluted with deionized RNase-free water (to 10 mL), added to the top of and eluted (60% methanol-H₂O) through a short C18 cartridge (Sep-Pak C18). UV active fractions were collected, combined and concentrated till dryness in a Savant™ SpeedVac™ vacuum concentrator at ambient temperature overnight. The sample was submitted for HPLC/LC-MS analysis as a solid.

### Example 11: Synthesis of an 12gRNA Construct Containing a Non-Nucleotide Linker in the Spacer (9 nt+23 nt)

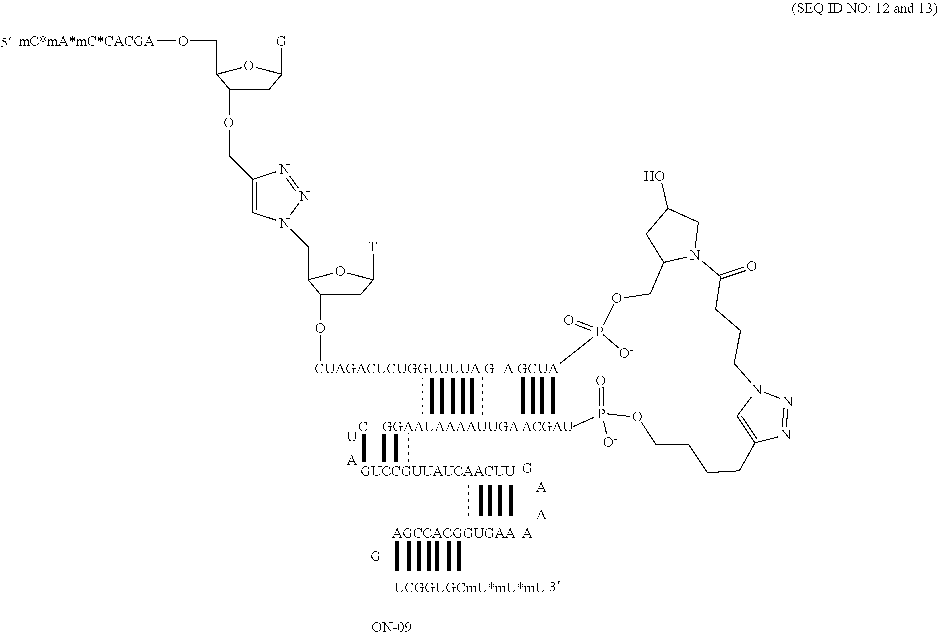

Spacer segment 1 (5′-mC*mA*mC*CACGAG-propargyl-3′) was synthesized on an Expedite 8909 using 3′-propargyl guanosine 2′-lcaa CPG 1000 Å (1 μmole). Coupling efficiencies were >98%. Deprotection and precipitation were similar to EXAMPLE 1.

Synthesis of Spacer Segment 2 (crRNA-3′):

Sequence:
(ON-10, SEQ ID NO: 12)
5′-amino-TCUAGACUCUGGUUUUAGAGCUA-N₃-3′
(5′-amino, 3′-azido modification)

Prepared on an Expedite 8909 automated DNA/RNA synthesizer using a standard 1.0 μmole RNA phosphoramidite cycle. 3′-Azido CPG 1000 Å (1 μmole) was packed into an Expedite column. β-Cyanoethyl phosphoramidite monomers (0.1 M in anhydrous acetonitrile) were coupled with 5-Ethyl-1H-tetrazole (0.45 M in acetonitrile), capped with Cap A (Acetic Anhydride/Pyridine/THF) and Cap B (10% N-Methylimidazole in THF), and oxidized with iodine (0.02 M Iodine/Pyridine/H₂O/THF). Coupling efficiencies exceeded 98%, monitored by trityl cation conductivity.

The oligonucleotide on solid support in the reaction column was treated with 20% piperidine in DMF at room temperature for 30 min to suppress cyanoethyl adducts, washed with acetonitrile (3×1 mL), and dried with argon. Detritylation of 5′-dT was performed using 3% TCA/DCM, and the column was further washed with acetonitrile (3×1 mL), and dried under vacuum. Deprotection with AMA (Ammonium Hydroxide/40% aqueous Methylamine 1:1 v/v) at 65° C. for 20 min, filtered, and concentrated to dryness in a Savant™ SpeedVac™ vacuum concentrator at room temperature. The solid was redissolved in NMP/triethylamine/triethylamine trihydrofluoride (2:2:3 v/v, 700 μL total) and heated at 60° C. for 3 h. After cooling, sodium acetate (3 M, pH 5.2, 40 μL) and ethanol (1 mL) were added, cooled to −78° C. for 30 min, centrifuged (15,850×g, 10 min, 4° C.), washed with 70% ethanol (2×500 μL), and dried in vacuo—yielding crRNA-3′.

Synthesis of Spacer Segment 2 with tracrRNA (5′-amino-crRNA-3′-tracrRNA):

5′-Hexynyl-trarcRNA was synthesized on an Expedite 8909 using 2′-methoxy uridine 3′-lcaa CPG 1000 Å (1 μmole) similar to EXAMPLE 2.

Ligation: Spacer Segment 2 (0.5 μmole) and 5′-Hexynyl-tracrRNA (0.5 μmole) in DMSO/ddH₂O/2 M TEAA (2:1:0.4, 1700 μL) were mixed with CuSO₄-THPTA (250 mM, 100 μL), deoxygenated with argon for 10 min, then treated with ascorbic acid (125 mM, 200 μL) and further deoxygenated for 30 min. The mixture was sealed, reacted at room temperature for 2 h, precipitated with sodium acetate (3 M, pH 5.2, 40 μL) and ethanol (1 mL) at −78° C. for 30 min, centrifuged, washed with 70% ethanol (2×500 μL), and dried—yielding 5′-amino-crRNA-3′-tracrRNA.

Final Ligation to Form 12gRNA (ON-09):

5′-amino-crRNA-3′-tracrRNA was transformed into 5′-azido-crRNA-3′-tracrRNA using a reported procedure via a diazotransfer reaction (Zhong, WO2023101993, the entire disclosures of which are incorporated herein by reference.).

crRNA-propargyl-5′ (˜0.5 μmole) and 5′-azido-crRNA-3′-tracrRNA (˜0.5 μmole) were dissolved in DMSO/ddH₂O/2 M TEAA (2:1:0.4, 1700 μL), mixed with CuSO₄-THPTA (250 mM, 100 μL), deoxygenated with argon for 10 min, then treated with ascorbic acid (125 mM, 200 μL) and deoxygenated for 30 min. The reaction proceeded at room temperature for 2 h, followed by precipitation with sodium acetate (3 M, pH 5.2, 40 μL) and ethanol (1 mL) at −78° C. for 30 min, centrifugation (15,850×g, 10 min, 4° C.), washing with 70% ethanol (2×500 μL), and drying in vacuo.

Purification: The pellet was dissolved in gel loading buffer (90% formamide/ddH₂O with 10 mM EDTA), mixed with RNA loading dyes (2x), and separated on a denaturing 10% polyacrylamide gel (1x TBE, 7 M urea) at 65 W for 2-3 h. RNA bands were visualized under UV, excised, crushed, and soaked in gel extract buffer (NaCl with 2 mM EDTA) overnight at 37° C. with shaking. The extract was filtered through two Sep-Pak C18 cartridges, eluted with 60% methanol-H₂O, quantified at 260 nm via NanoDrop, and concentrated—yielding 12gRNA (ON-08).

Alternatively, L2gRNA can be synthesized by ligation between a ligated 3′-amino-crRNA (containing an internal triazole linker) and a 5′-alkynyl-tracrRNA via click chemistry of an azido derived from the 3′-amino by either diazotransfer reaction or NHS-mediated selective amide formation (Zhong, WO2023101993, the entire disclosures of which are incorporated herein by reference.).

L2gRNA comprising a ligated spacer of:

5′-mC*mA*mC*CACGAGUCtzUAGACUCUGGUUUUAGAGCUA-3′
(11nt + 21nt, ON-12 (SEQ ID NO: 14 and 15)),
5′-mC*mA*mC*CACGAGUtzCUAGACUCUGGUUUUAGAGCUA-3′
(10nt + 22nt, ON-13 (SEQ ID NO: 16 and 17)),
or
5′-mC*mA*mC*CACGAGUCUtzAGACUCUGGUUUUAGAGCUA-3′
(12nt + 20nt, ON-14 (SEQ ID NO: 18 and 19))

with optional chemical modifications can be synthesized similarly, wherein tz is a triazole linker.

What is claimed is:

1. Constructs of a guide RNA comprising a non-nucleotide linker in its spacer.

2. Said constructs of a guide RNA of claim 1, wherein said non-nucleotide linker is a triazole linker selected from the group consisting of nNt-linker-1, nNt-linker-2, nNt-linker-3 and nNt-linker-4: 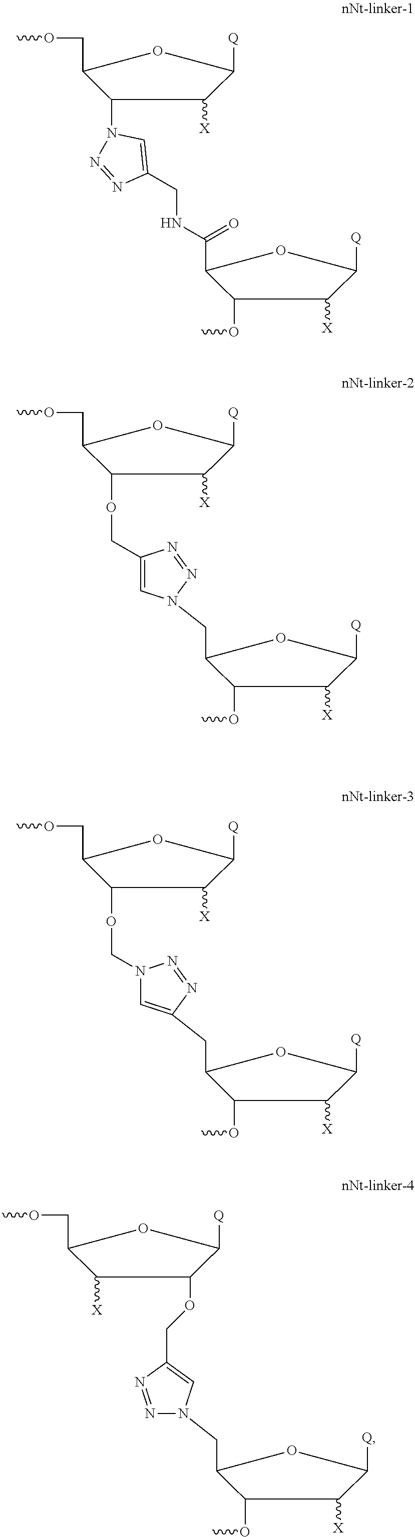
, wherein the two nucleotides joined by said non-nucleotide linker are selected from the spacer sequence, two Qs are the nucleic acid bases, and X is selected from the group consisting of H, F, Cl, low alkyl of C1-C6, OH, OMe, OR, OMOE, NH₂ and NHR and can be in either α- or β-configuration, or is covalently linked to 4′-position to form a locked nucleoside, wherein R is an acyl, alkyl, or aryl, and the two Xs can be the same or different.

3. Said constructs of a guide RNA of claim 1, wherein the spacer has a non-nucleotide linker between positions 6 and 7, 7 and 8, 8 and 9, 9 and 10, 10 and 11, 11 and 12 or 12 and 13 (numbered from the 5′-end of the spacer).

4. Said constructs of a guide RNA of claim 1, wherein said guide is a lgRNA comprising crRNA of said spacer of claim 1 and tracrRNA joined by a nucleotide linker, wherein said tracrRNA optionally comprises one or more non-nucleotide linkers.

5. Said constructs of a guide RNA of claim 1, wherein said guide is a lgRNA comprising crRNA of said spacer of claim 1 and tracrRNA joined by a non-nucleotide linker, wherein said tracrRNA optionally comprises one or more non-nucleotide linkers.

6. Said constructs of a guide RNA of claim 1, wherein said guide is crRNA.

7. Said constructs of a guide RNA of claim 1, comprising crRNA of said spacer of claim 1, tracrRNA, a prime binding sequence (PBS) and a reverse transcription template (RTT) and an optional 3′-end stabilizing RNA motif, and optionally comprising one or more non-nucleotide linkers.

8. Said constructs of a guide RNA of claim 1, comprising crRNA of said spacer of claim 1, tracrRNA and an ssDNA template, and optionally comprising one or more non-nucleotide linkers.

9. Constructs of a guide RNA comprising a restriction site.

10. Said constructs of a guide RNA of claim 9, where the restriction site is a EcoRI recognition sequence (gaattc).

11. Said constructs of a guide RNA of claim 9, wherein said guide is a sgRNA comprising crRNA and tracrRNA joined by a nucleotide linker.

12. Said constructs of a guide RNA of claim 9, wherein said guide is a lgRNA comprising crRNA and tracrRNA joined by a non-nucleotide linker, wherein said tracrR NA optionally comprises one or more non-nucleotide linkers.

13. Said constructs of a guide RNA of claim 9, wherein said guide is a crRNA.

14. Said constructs of a guide RNA of claim 9, comprising crRNA, tracrRNA, a prime binding sequence (PBS) and a reverse transcription template (RTT) and an optional 3′-end stabilizing RNA motif, and optionally comprising one or more non-nucleotide linkers.

15. Said constructs of a guide RNA of claim 9, comprising crRNA, tracrRNA and an ssDNA template, and optionally comprising one or more non-nucleotide linkers.

16. A method to quality control guide RNA products by analyses of a short spacer fragment formed by treatment of the guide RNA constructs with a restriction enzyme.

17. Said method of claim 16, wherein said short spacer fragment has a length of 20-30 nt.

18. Said method of claim 16, wherein said short spacer fragment has a length of 30-40 nt.

19. Said method of claim 16, wherein said short spacer fragment has a length of 40-50 nt.

20. Said method of claim 16, wherein said short spacer fragment has a length of 50-60 nt.


SEQUENCE LISTING (23 sequences)

SEQ ID NO: 1 - RNA, 22 nt, synthetic construct
  misc_difference 1..10: note = n is a, c, g or u.
  source 1..22: mol_type = other RNA
  modified_base 1: mod_base = OTHER; note = 5'-triazol-1-yl-5'-deoxynucelotide. 1 is joined to the 3'-end of other segment of spacer at its 5'-end via trizaole.
nnnnnnnnnngttttagagcta

SEQ ID NO: 2 - RNA, 64 nt, synthetic construct
  source 1..64: mol_type = other RNA
  misc_feature 1: note = 1 is joined to the 3'-end of SEQ ID NO 1 via a non-nucleotide linker at its 5'-end.
tagcaagttaaaataaggctagtccgttatcaacttgaaaaagtggcaccgagtcggtgctttt

SEQ ID NO: 3 - RNA, 112 nt, synthetic construct
  source 1..112: mol_type = other RNA
  misc_feature 33..48: note = gaattcGAAAgaattc is EcoRI restriction site.
nnnnnnnnnnnnnnnnnnnngttttagagctagaattcgaaagaattctagcaagttaaaataaggctagtccgttatcaacttgaaaaagtggcaccgagtcggtgctttt

SEQ ID NO: 4 - RNA, 38 nt, synthetic construct
  source 1..38: mol_type = other RNA
  misc_feature 33..38: note = gaattc is a restriction site. 38 is joined to the 5'-end of SEQ ID NO 5 at its 3'-position via a non-nucleotide linker.
nnnnnnnnnnnnnnnnnnnngttttagagctagaattc

SEQ ID NO: 5 - RNA, 70 nt, synthetic construct
  source 1..70: mol_type = other RNA
  modified_base 1: mod_base = OTHER; note = 2'-deoxyguanosine 3'-phosphorothioate. 1 is joined to the 3'-end of SEQ ID NO 4 at its 5'-position via a non-nucleotide linker.
  modified_base 2..3: mod_base = OTHER; note = 2'-deoxyadenosine 3'-phosphorothioate.
  modified_base 4..5: mod_base = OTHER; note = thymidine 3'-phosphorothioate.
  modified_base 6: mod_base = OTHER; note = 2'-deoxycytidine 3'-phosphorothioate.
gaattctagcaagttaaaataaggctagtccgttatcaacttgaaaaagtggcaccgagtcggtgctttt

SEQ ID NO: 6 - RNA, 108 nt, synthetic construct
  source 1..108: mol_type = other RNA
  misc_feature 108: note = 3'-end (108) is extended with an RNA template and an optional stabilizing motif for prime editing.
  misc_feature 33..48: note = gaattcGAAAgaattc is EcoRI restriction site.
nnnnnnnnnnnnnnnnnnnngttttagagctagaattcgaaagaattctagcaagttaaaataaggctagtccgttatcaacttgaaaaagtggcaccgagtcggtgc

SEQ ID NO: 7 - RNA, 108 nt, synthetic construct; identical to SEQ ID NO: 6
  source 1..108: mol_type = other RNA
  misc_feature 108: note = 3'-end (108) is extended with an ssDNA (at either its 3'- or 5'-end).
  misc_feature 33..48: note = gaattcGAAAgaattc is EcoRI restriction site.

SEQ ID NO: 8 - RNA, 38 nt, synthetic construct
  source 1..38: mol_type = other RNA
  modified_base 1: mod_base = OTHER; note = 2'-methoxycytidine 3'-phosphorothioate
  modified_base 2: mod_base = OTHER; note = 2'-methoxyuridine 3'-phosphorothioate
  modified_base 3: mod_base = OTHER; note = 2'-methoxycytidine 3'-phosphorothioate
  modified_base 33: mod_base = OTHER; note = 2'-deoxyguanosine 3'-phosphorothioate.
  modified_base 34..35: mod_base = OTHER; note = 2'-deoxyadenosine 3'-phosphorothioate.
  modified_base 36..37: mod_base = OTHER; note = thymidine 3'-phosphorothioate.
  modified_base 38: mod_base = OTHER; note = 2'-deoxycytidine 3'-phosphorothioate. 1 is joined with a conjugtaion functional group (azido) via its phosphorothioate ester or joined to the 5'-end of SEQ ID NO 9 at its 3'-position via a non-nucleotide linker.
ctcgtgaccaccctgacctagttttagagctagaattc

SEQ ID NO: 9 - RNA, 69 nt, synthetic construct
  source 1..69: mol_type = other RNA
  modified_base 67: mod_base = OTHER; note = 2'-methoxyuridine 3'-phosphorothioate
  modified_base 68: mod_base = OTHER; note = 2'-methoxyuridine 3'-phosphorothioate
  modified_base 69: mod_base = um
  misc_feature 1: note = 1 is joined with a conjugtaion functional group via its 5'-phosphate diester or joined to the 3'-end of SEQ ID NO 8 at its 5'-position via a non-nucleotide linker.
gaattctagcaagttaaaataaggctagtccgttatcaacttgaaaaagtggcaccgagtcggtgcttt

SEQ ID NO: 10 - protein, 16 aa, synthetic construct
GGGRRRRRRRRRLLLL

SEQ ID NO: 11 - skipped

SEQ ID NO: 12 - RNA, 23 nt, synthetic construct
  source 1..23: mol_type = other RNA
  modified_base 1: mod_base = OTHER; note = 5'-triazol-1-yl-5'-deoxythymidine. 1 is joined with the other segment of spacer (mC*mA*mC*CACGAG) at its 3'-position via a non-nucleotide linker.
tctadactctggttttagagcta

SEQ ID NO: 13 - RNA, 63 nt, synthetic construct
  source 1..63: mol_type = other RNA
  modified_base 61: mod_base = OTHER; note = 2'-methoxyuridine 3'-phosphorothioate
  modified_base 62: mod_base = OTHER; note = 2'-methoxyuridine 3'-phosphorothioate
  modified_base 63: mod_base = um
  misc_feature 1: note = 1 is joined to the 3'-end of SEQ ID NO 12 at its 5'-position via a non-nucleotide linker.
tagcaagttaaaataaggctagtccgttatcaacttgaaaaagtggcaccgagtcggtgcttt

SEQ ID NO: 14 - RNA, 11 nt, synthetic construct
  source 1..11: mol_type = other RNA
  modified_base 1: mod_base = OTHER; note = 2'-methoxycytidine 3'-phosphorothioate
  modified_base 2: mod_base = OTHER; note = 2'-methoxyadenosine 3'-phosphorothioate
  modified_base 3: mod_base = OTHER; note = 2'-methoxycytidine 3'-phosphorothioate
  misc_feature 11: note = 11 is joined to the 5'-end of SEQ ID NO 15 at its 3'-position via a non-nucleotide linker.
caccacgagtc

SEQ ID NO: 15 - RNA, 21 nt, synthetic construct
  source 1..21: mol_type = other RNA
  misc_feature 1: note = 1 is joined to the 3'-end of SEQ ID NO 14 at its 5'-position via a non-nucleotide linker.
tagactctggttttagagcta

SEQ ID NO: 16 - RNA, 10 nt, synthetic construct
  source 1..10: mol_type = other RNA
  modified_base 1: mod_base = OTHER; note = 2'-methoxycytidine 3'-phosphorothioate
  modified_base 2: mod_base = OTHER; note = 2'-methoxyadenosine 3'-phosphorothioate
  modified_base 3: mod_base = OTHER; note = 2'-methoxycytidine 3'-phosphorothioate
  misc_feature 10: note = 10 is joined to the 5'-end of SEQ ID NO 17 at its 3'-position via a non-nucleotide linker.
caccacgagt

SEQ ID NO: 17 - RNA, 22 nt, synthetic construct
  source 1..22: mol_type = other RNA
  misc_feature 1: note = 1 is joined to the 3'-end of SEQ ID NO 16 at its 5'-position via a non-nucleotide linker.
ctagactctggttttagagcta

SEQ ID NO: 18 - RNA, 12 nt, synthetic construct
  source 1..12: mol_type = other RNA
  modified_base 1: mod_base = OTHER; note = 2'-methoxycytidine 3'-phosphorothioate
  modified_base 2: mod_base = OTHER; note = 2'-O-methyladenosine 3'-phosphorothioate
  modified_base 3: mod_base = OTHER; note = 2'-methoxycytidine 3'-phosphorothioate
  misc_feature 12: note = 12 is joined to the 5'-end of SEQ ID NO 19 at its 3'-position via a non-nucleotide linker.
caccacgagtct

SEQ ID NO: 19 - RNA, 20 nt, synthetic construct
  source 1..20: mol_type = other RNA
  misc_feature 1: note = 1 is joined to the 3'-end of SEQ ID NO 18 at its 5'-position via a non-nucleotide linker.
agactctggttttagagcta

SEQ ID NO: 20 - RNA, 102 nt, synthetic construct
  source 1..102: mol_type = other RNA
  misc_feature 33..38: note = gaattc is a restriction site.
nnnnnnnnnnnnnnnnnnnngttttagagctagaattctagcaagttaaaataaggctagtccgttatcaacttgaaaaagtggcaccgagtcggtgctttt

SEQ ID NO: 21 - RNA, 38 nt, synthetic construct; identical to SEQ ID NO: 4
  source 1..38: mol_type = other RNA
  misc_feature 33..38: note = gaattc is a restriction site. 38 is joined to the 5'-end of SEQ ID NO 22 at its 3'-position via a non-nucleotide linker.

SEQ ID NO: 22 - RNA, 70 nt, synthetic construct; identical to SEQ ID NO: 5
  source 1..70: mol_type = other RNA
  misc_feature 1..6: note = gaattc is a restriction site. 1 is joined to the 3'-end of SEQ ID NO 21 at its 5'-position via a non-nucleotide linker.

SEQ ID NO: 23 - RNA, 38 nt, synthetic construct; identical to SEQ ID NO: 4
  source 1..38: mol_type = other RNA
  modified_base 33: mod_base = OTHER; note = 2'-deoxyguanosine 3'-phosphorothioate.
  modified_base 34..35: mod_base = OTHER; note = 2'-deoxyadenosine 3'-phosphorothioate.
  modified_base 36..37: mod_base = OTHER; note = thymidine 3'-phosphorothioate.
  modified_base 38: mod_base = OTHER; note = 2'-deoxycytidine 3'-phosphorothioate. 38 is joined to the 5'-end of SEQ ID NO 22 at its 3'-position via a non-nucleotide linker.